(19) United States
(12) Patent Application Publication
(10) Pub. No.: US 2025/0072924 A1
(43) Pub. Date: Mar. 6, 2025

(54) APPARATUS AND METHODS OF TRIGGER DIGIT RELEASE

(71) Applicant: Alton Lee Hunter, JR., Columbia, TN (US)

(72) Inventor: Alton Lee Hunter, JR., Columbia, TN (US)

(21) Appl. No.: 18/751,097

(22) Filed: Jun. 21, 2024

Related U.S. Application Data
(60) Provisional application No. 63/536,146, filed on Sep. 1, 2023.

(51) Int. Cl. A61B 17/32; A61B 17/00

(52) U.S. Cl. CPC A61B 17/320016; A61B 2017/00238; A61B 2017/00336; A61B 2017/00424; A61B 2017/00738

(57) ABSTRACT

A tissue dissection apparatus for treatment of a trigger digit is provided. The apparatus may include a handle, a shaft extending distally from the handle, a receiver defined in a proximal end of the handle opposite the shaft, and a foot disposed on a distal end of the shaft opposite the handle. The receiver may include a port shaped to accommodate entry of a cutting instrument such as a needle. The foot may include a concave contact surface and an opening disposed on the concave contact surface. The concave contact surface may be shaped to approximate the contour of a tendon sheath. The apparatus may further include a channel defined from the port to the opening. The channel may be configured to axially receive the cutting instrument including a tip that is configured to dissect tissue during a surgical procedure.

### CROSS-REFERENCE TO RELATED APPLICATION

This application is a non-provisional of and claims priority to and benefit of U.S. Provisional Patent Application No. 63/536,146, filed Sep. 1, 2023, entitled Apparatus and Methods for Trigger Digit Release, which is hereby incorporated by reference in its entirety.

### BACKGROUND

The present disclosure relates generally to devices and methods for performing surgery and more particularly to devices and methods for dissecting tissue in a human hand.

Finger and thumb tendonitis, also known as trigger finger or trigger digit, is one of the most common orthopedic conditions. Trigger digit occurs when a finger or thumb is unable to fully flex and extend relative to the hand. In many cases, the finger or thumb becomes partially bent toward the palm of the hand, and a patient is unable to straighten the digit. Trigger digit causes pain and stiffness in the hand and fully disables a patient's ability to use the digit in some cases.

In cases of trigger digit, the flexor tendon that causes a finger or thumb to flex is unable to freely travel through a corresponding tendon sheath or a tendon pulley. In some instances, the A1 pulley in a patient's hand becomes inflamed or thickened, or the flexor tendon itself becomes inflamed or thickened, thereby restricting travel of the flexor tendon and preventing proper flexion or extension of a digit.

Trigger digit release is one of the most common treatments provided by orthopedic surgeons. Treatment options for trigger finger or trigger thumb include splinting (rarely successful), physical therapy (almost never successful), cortisone injections (less than 50% success rate) and surgery. Percutaneous trigger digit releases are performed in clinical offices by surgeons in an office setting, saving the patient the inconvenience and expense of a formal “open” surgical procedure. Historically, percutaneous trigger digit releases have been performed in clinical offices by a select few orthopedic surgeons, using a variety of techniques with a highly variable success rate (at best) of 90% with regards to eliminating the mechanical symptoms of “catching” in the digit and pain. Traditional “open” surgery has a success rate of better than 99%, thus the vast majority of trigger digit release operations are performed in this manner, requiring a surgical facility along with the added expense and inconvenience associated with it.

Trigger digit can be treated in many cases by severing, or dissecting, the A1 pulley to free the affected flexor tendon, known as a trigger digit release operation. Trigger digit release surgery can be performed in some instances by percutaneously inserting a needle (e.g., a spinal needle, or other suitable needle) into the palm of the hand and using the sharpened tip of the needle as a cutting tool to sever the A1 pulley of the affected digit. Such operations may be performed in a doctor's office without general anesthesia. However, conventional methods of percutaneous trigger digit release using a needle may be difficult for some surgeons to perform because the needle tip cannot be directly observed in real time during the operation, and many surgeons are not comfortable guiding the needle tip to the A1 pulley percutaneously based on feel. For this reason, most surgeons choose to perform trigger release operations in open surgery, forming an incision in the palm to gain direct access to and visual observation of the A1 pulley. However, such operations must be performed in a surgical suite and may require anesthesia. This leads to much greater expense and a longer recovery time for the patient.

What is needed are improvements in devices and methods for percutaneous trigger digit release such that operations may be performed safely, reliably, and repeatably in a surgeon's office as opposed to a surgical suite.

### BRIEF SUMMARY

The present disclosure provides a surgical tool for dissecting, or cutting, tissue. In some embodiments, the surgical tool is configured for performing an operation to treat trigger digit in a finger or thumb by dissecting the A1 pulley to free a constrained flexor tendon. The surgical tool operates as a guide for steering a needle to safely, precisely, and reliably dissect tissue by a surgeon in either a clinical office or in a surgical suite.

In some embodiments, the surgical tool provides an apparatus including a handle; a shaft extending distally from the handle; a receiver defined in a proximal end of the handle opposite the shaft; a foot disposed on a distal end of the shaft opposite handle; and a channel defined through the handle and at least a portion of the shaft.

An instrument such as a needle may be inserted into the receiver and through the shaft such that a hub of the needle is seated in the receiver, thereby securing the needle at a desired angular orientation about its longitudinal axis. A tip of the needle protrudes from the shaft distal to the foot such that the foot may be inserted in a small incision and the tip of the needle may be precisely advanced to dissect tissue by manually controlling the handle in a back and forth or side to side motion. The foot includes a contact surface shaped to align with a flexor tendon sheath in some embodiments. The foot may serve as a soft tissue dissector tool, retractor tool, and a safety mechanism by limiting the depth of the deployed cutting needle and thus preventing damage to the flexor tendon. Once aligned with the A1 pulley, the needle tip may be used to cut the tissue at a desired location. The entire procedure may be performed with or without ultrasound imaging in a doctor's office. The “foot” serves as a soft tissue dissector tool, retractor tool, and a safety mechanism by limiting the depth of the deployed cutting needle and thus preventing damage to the flexor tendon.

One object of the present disclosure is to provide an effective, predictable, safe and technically easy percutaneous trigger digit release tool.

Another object of the present disclosure is to provide a trigger digit release tool compatible for use with ultrasound imaging in a doctor's office as opposed to open surgery.

A further object of the present disclosure is to provide a trigger digit release tool deployable with a small incision that does not require suturing.

Another object of the present disclosure is to provide a trigger digit release tool with a foot that limits depth of cut to limit potential damage to the flexor tendon.

A further object of the present disclosure is to provide a trigger digit release tool with a bidirectional working end that may be utilized using a push or a pull cutting technique.

An additional object of the present disclosure is to provide a trigger digit release tool with a foot having one or more inclined edges to dissect tissue while advancing.

A further object of the present disclosure is to provide a trigger digit release tool to utilize a common spinal needle deployed as a cutting instrument having precise depth control by manipulating the position of the needle relative to the tool.

Yet another object of the present disclosure is to provide a trigger digit release tool having a handle with curved upper surface and bottom surface to approximate the curvature of a flexor sheath to facilitate precise orientation and visualization of the cutting instrument relative to the anatomy of the hand.

A further object of the present disclosure is to provide a trigger digit release tool that is sterile and disposable.

Numerous other objects, advantages and features of the present disclosure will be readily apparent to those of skill in the art upon a review of the following drawings and description.

### BRIEF DESCRIPTION OF THE FIGURES

FIG. 1 illustrates a front perspective view of an embodiment of a surgical tool.

FIG. 2 illustrates a rear perspective view of an embodiment of a surgical tool.

FIG. 3 illustrates a rear perspective view of an embodiment of a surgical tool.

FIG. 4 illustrates a cross-sectional perspective view of an embodiment of a surgical tool.

FIG. 5 illustrates a perspective view of an embodiment of an instrument for cutting tissue including a needle with a hub and a tube.

FIG. 6 illustrates a cross-sectional perspective view of an embodiment of a surgical tool.

FIG. 7 illustrates an exploded perspective view of an embodiment of an embodiment of a surgical tool and an instrument for cutting tissue including a needle with a hub and a tube.

FIG. 8 illustrates a side view of an embodiment of a surgical tool including an instrument installed for cutting tissue.

FIG. 9 illustrates front elevation view of an embodiment of a surgical tool including an instrument installed for cutting tissue.

FIG. 10 illustrates a detail side elevation view of an embodiment of a shaft and a foot of a surgical tool.

FIG. 11 illustrates a front elevation view of an embodiment of a shaft and a foot of a surgical tool.

FIG. 12 illustrates a detail perspective view of an embodiment of an instrument including a tube and a needle tip.

FIG. 13 illustrates a side elevation cross-sectional view of an embodiment of a surgical tool.

FIG. 14 illustrates a front view of an embodiment of a surgical tool tilted away from a reference vertical plane.

FIG. 15 illustrates a detail top view of an embodiment of a shaft and foot of a surgical tool.

FIG. 16 illustrates a perspective view of an embodiment of a surgical tool including a straight shaft.

FIG. 17 illustrates a perspective cross-sectional view of an embodiment of a surgical tool including a straight shaft.

FIG. 18 illustrates an upper-rear perspective view of an embodiment of a surgical tool.

FIG. 19 illustrates an upper-front perspective view of an embodiment of a surgical tool.

FIG. 20 illustrates a detailed perspective view of an embodiment of a shaft and a foot of a surgical tool.

FIG. 21 illustrates a front view of an embodiment of a surgical tool.

FIG. 22 illustrates a detailed top view of an embodiment of a shaft and a foot of a surgical tool.

FIG. 23 illustrates a side view of an embodiment of a surgical tool.

FIG. 24 illustrates a top view of an embodiment of a surgical tool.

FIG. 25 illustrates a lower view of an embodiment of a surgical tool.

FIG. 26 illustrates a side cross-sectional view of an embodiment of a surgical tool.

FIG. 27 illustrates a detailed side cross-sectional view of an embodiment of a surgical tool.

FIG. 28 illustrates a side view of an embodiment of an instrument for cutting tissue including a needle with a hub and a tube.

FIG. 29 illustrates an upper-front perspective view of an embodiment of a surgical tool including an instrument installed for cutting tissue.

FIG. 30 illustrates an upper-rear perspective view of an embodiment of a surgical tool including an instrument installed for cutting tissue.

FIG. 31 illustrates a side cross-sectional view of an embodiment of a surgical tool including an instrument installed for cutting tissue.

FIG. 32 illustrates a detailed front perspective view of an embodiment of a shaft and a foot of a surgical tool with an instrument installed for cutting tissue.

FIG. 33 illustrates a detailed upper perspective view of an embodiment of a shaft and a foot of a surgical tool with an instrument installed for cutting tissue.

FIG. 34 illustrates a detailed top view of an embodiment of a shaft and a foot of a surgical tool with an instrument installed for cutting tissue.

FIG. 35 illustrates a detailed top view of a handle of a surgical tool.

FIG. 36 illustrates a detailed side cross-sectional view of an embodiment of a shaft and a foot of a surgical tool.

FIG. 37 illustrates a rear view of an embodiment of a surgical tool.

FIG. 38 illustrates a lower-rear perspective view of an embodiment of a surgical tool.

FIG. 39 illustrates a side-rear perspective view of an embodiment of an instrument for cutting tissue including a needle with a hub and a tube.

FIG. 40 illustrates a side-rear perspective view of an embodiment of an instrument for cutting tissue including a needle with a hub and a tube.

FIG. 41 illustrates a detailed top view of an embodiment of a shaft and a foot of a surgical tool with an instrument installed in an opposite orientation relative to the instrument of FIG. 34.

FIG. 42 illustrates an exemplary embodiment of a method of treating a trigger digit, including the step of making a small incision in a hand.

FIG. 43 illustrates an exemplary embodiment of a method of treating a trigger digit, including the step of positioning a foot of a surgical tool over a tendon sheath.

FIG. 44 illustrates an exemplary embodiment of a method of treating a trigger digit, including the step of advancing an instrument for cutting tissue through the cutting tool.

FIG. 45 illustrates an exemplary embodiment of a method of treating a trigger digit, including the step of using a cutting instrument installed in a surgical tool to cut diseased tissue, in this case a soft tissue pulley.

FIG. 46 illustrates a detailed exemplary embodiment of a method of treating a trigger digit, including the step of using a cutting instrument installed in a surgical tool to cut a tendon.

### DETAILED DESCRIPTION

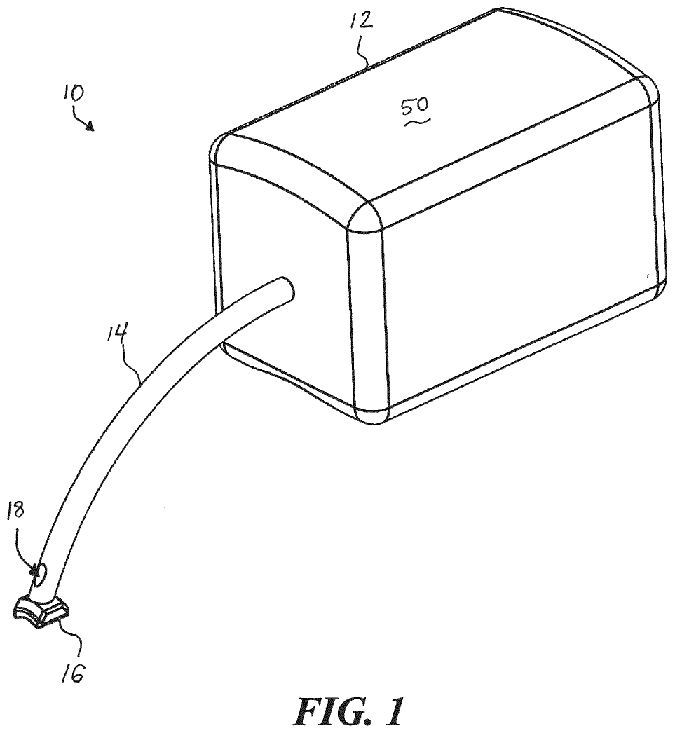

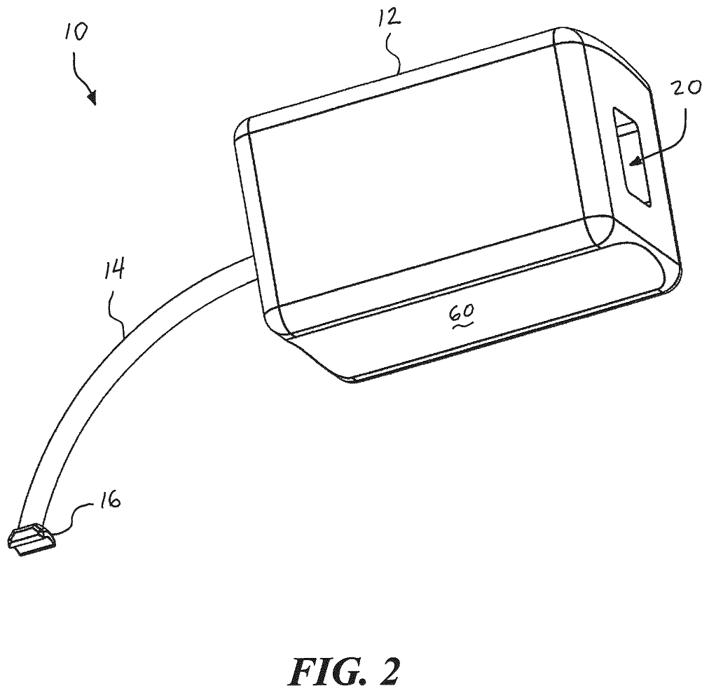

The present disclosure provides a tissue dissection apparatus and methods for treatment of trigger digit. Referring to FIGS. 1-4, in some embodiments, a surgical tool apparatus (apparatus) 10 includes a body having a handle 12 and a shaft 14 extending distally from the handle 12. A receiver 20 may be defined in a proximal end of the handle 12 opposite the shaft. A foot 16 may be disposed on a distal end of the shaft 14 opposite the handle 12. A channel 24 may be defined through the handle and at least partially through the shaft 14. The apparatus 10 may be configured such that handle 12 can be held in a user's hand with the shaft 14 extending toward an incision site for performing a trigger digit release surgical procedure in a surgeon's office.

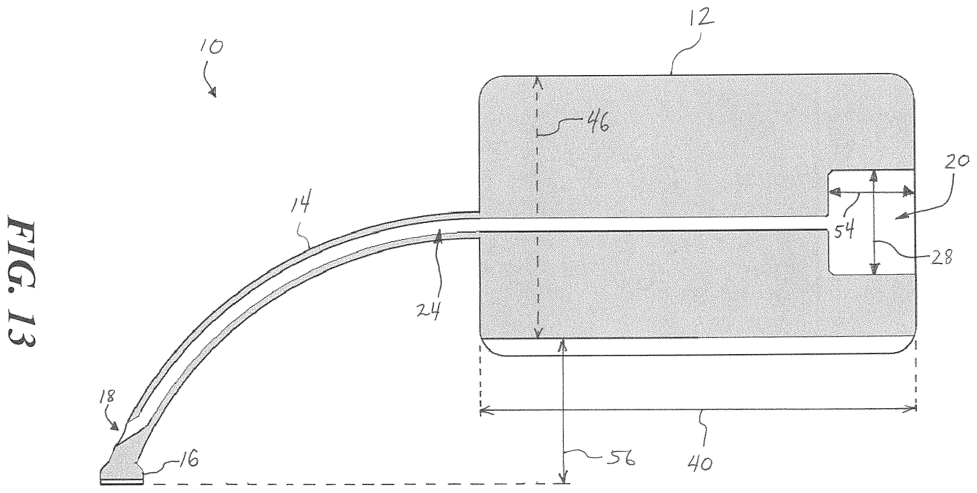

In some embodiments, handle 12 includes a member having a profile dimensioned such that handle 12 may be gripped in a user's hand. For example, the handle 12 may include a convex curved top surface 50 and a concave curved bottom surface 60. Depending on the implementation, the handle 12 may include a handle length 40 of about forty millimeters, a handle width of about thirty millimeters, and a handle height 46 of about thirty millimeters, as shown in FIG. 13. Handle 12 may include other shapes and dimensions in further embodiments.

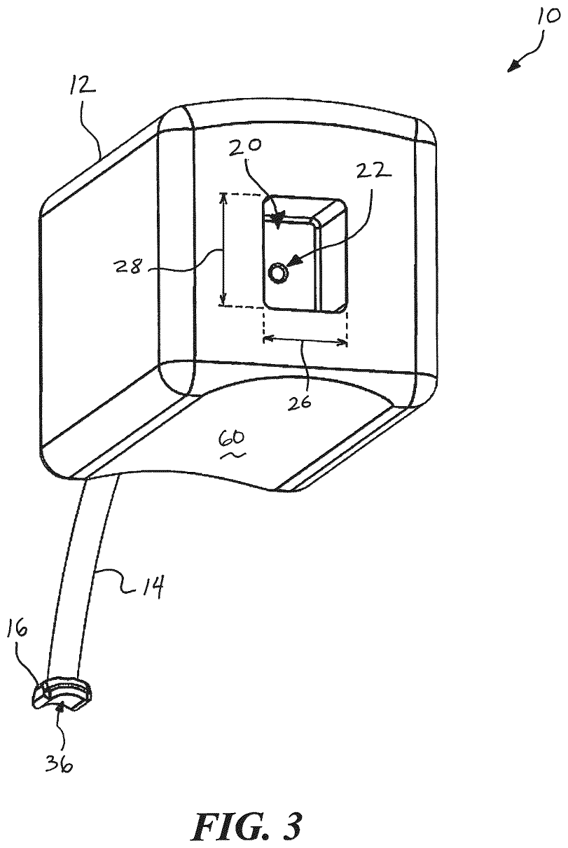

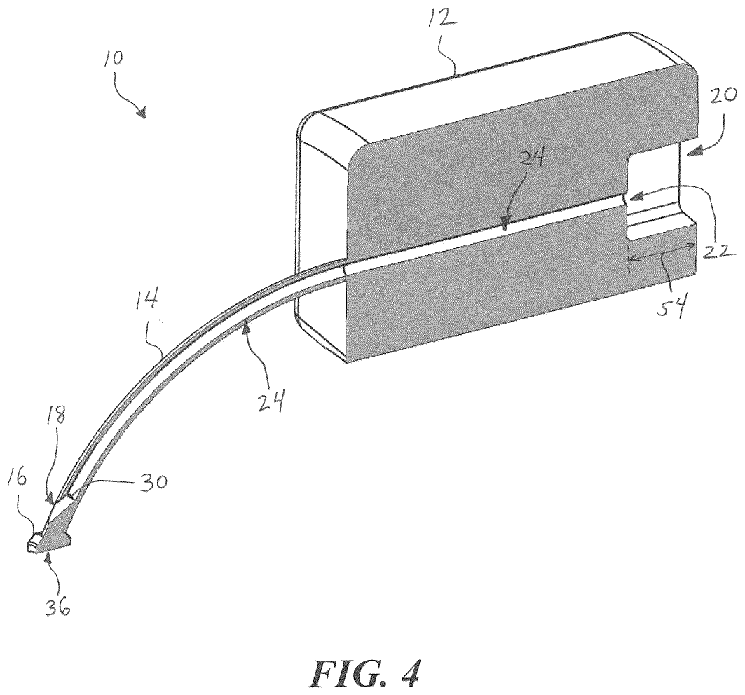

Referring to FIGS. 3 and 4, receiver 20 on handle 12 is defined on the proximal end of handle 12 opposite shaft 14, according to some embodiments of the present disclosure. Receiver 20 may include a recess formed in handle 12, which may be shaped and dimensioned to accommodate a feature on a cutting instrument, such as a hub on a needle. In this sense, receiver 20 may be integrally formed as a recess in handle 12. In other embodiments, receiver 20 includes an attachment or fitting on handle 12, which may be shaped and dimensioned to receive a portion of an instrument inserted into handle 12. Receiver 20 includes a rectangular profile including a receiver recess height 28, a receiver recess width 26 and a receiver recess depth 54 in some embodiments. In other embodiments, receiver 20 includes any suitable shape for receiving a portion of an instrument, such as but not limited to a hub on a needle. In some embodiments, receiver recess height 28 is about twelve millimeters, receiver recess width 26 is about nine millimeters, and receiver recess depth 54 is about ten millimeters.

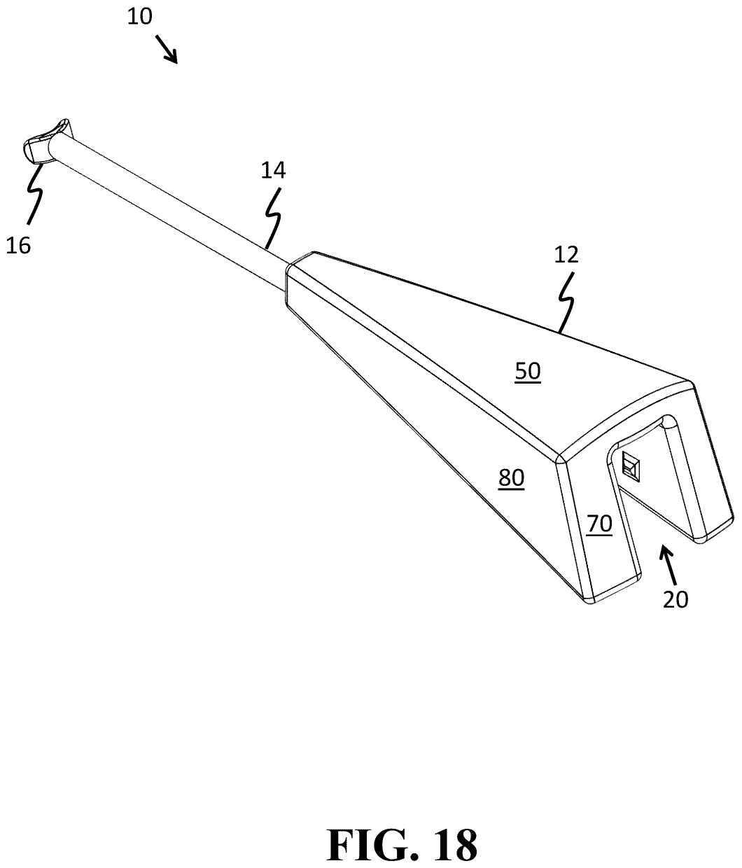

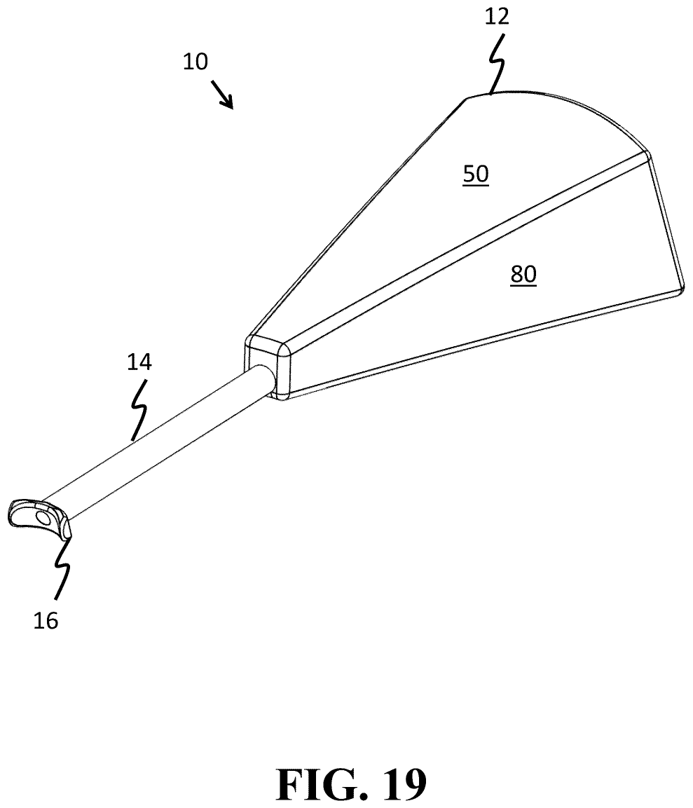

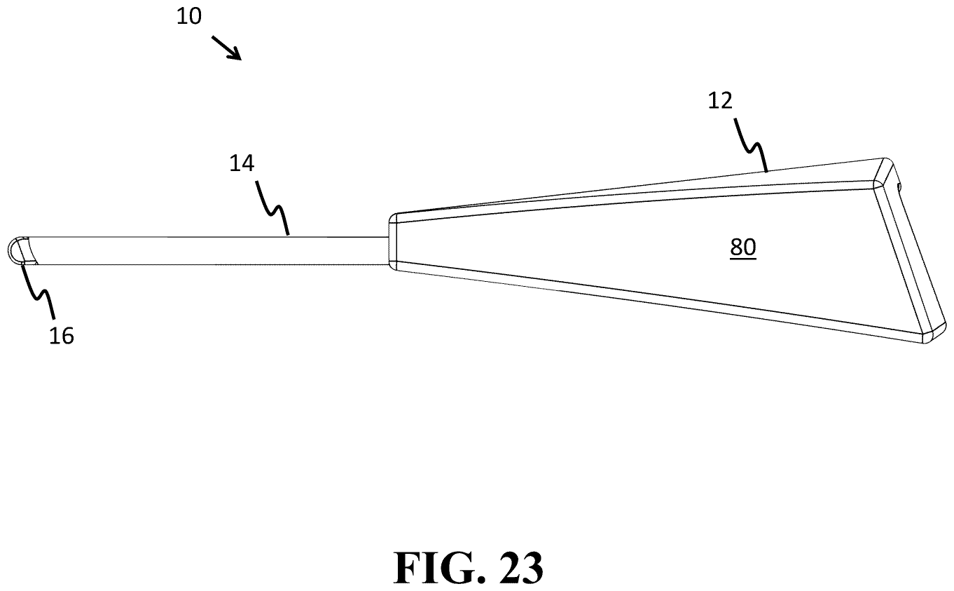

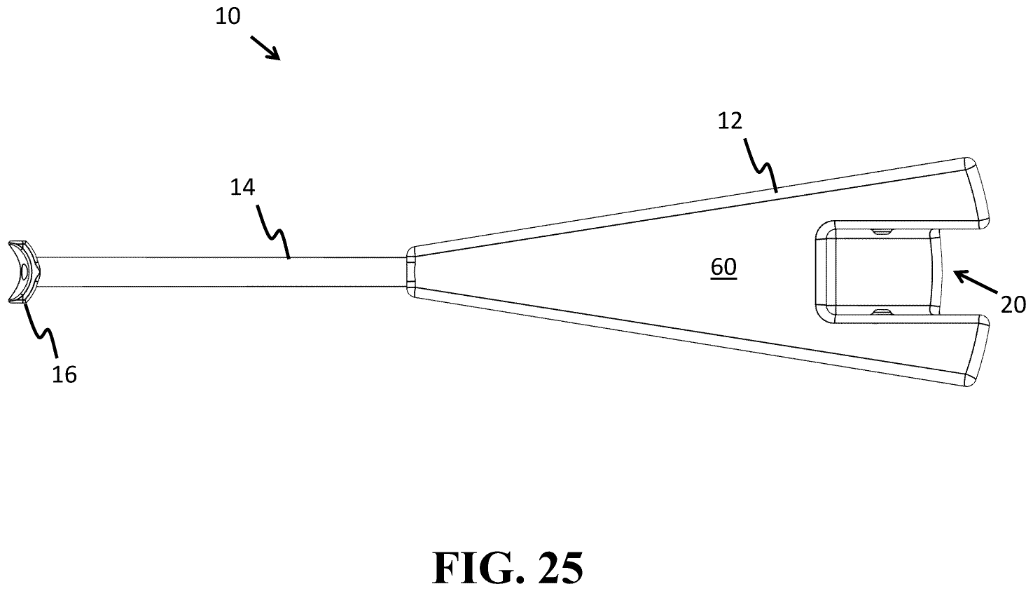

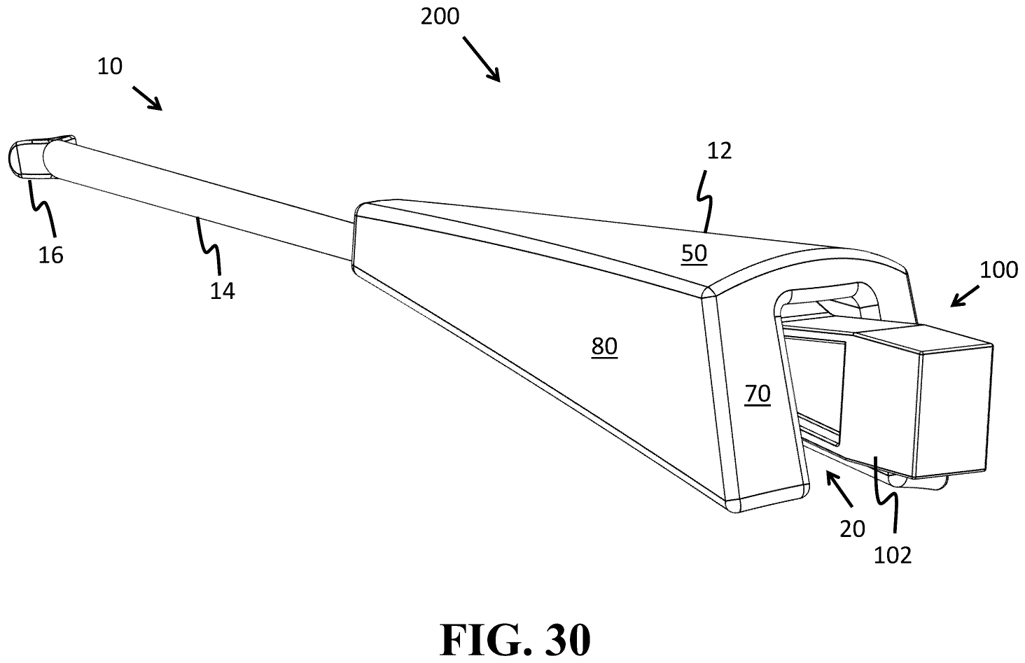

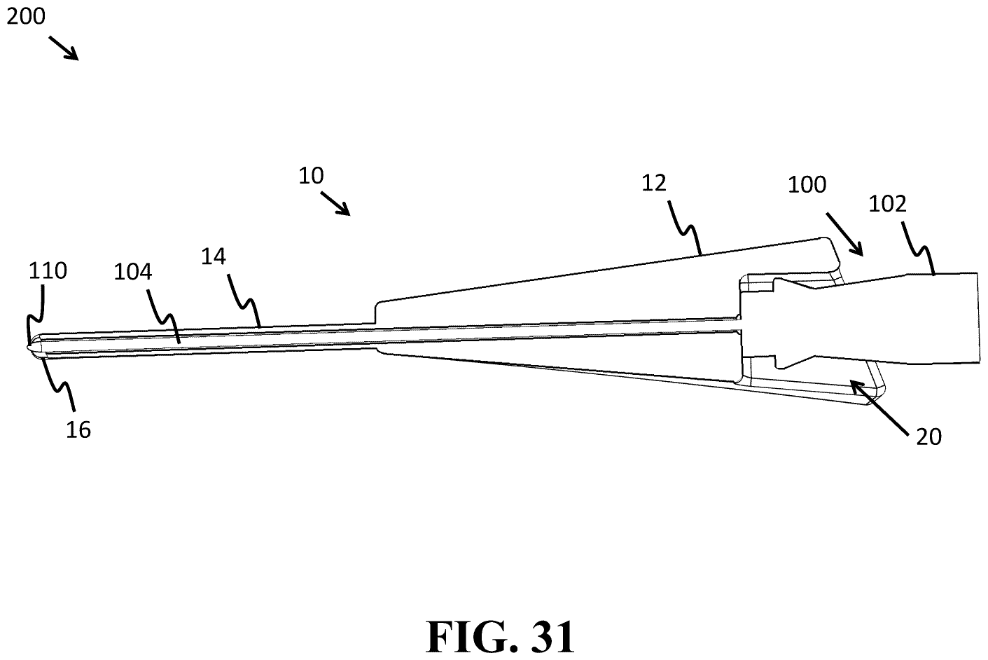

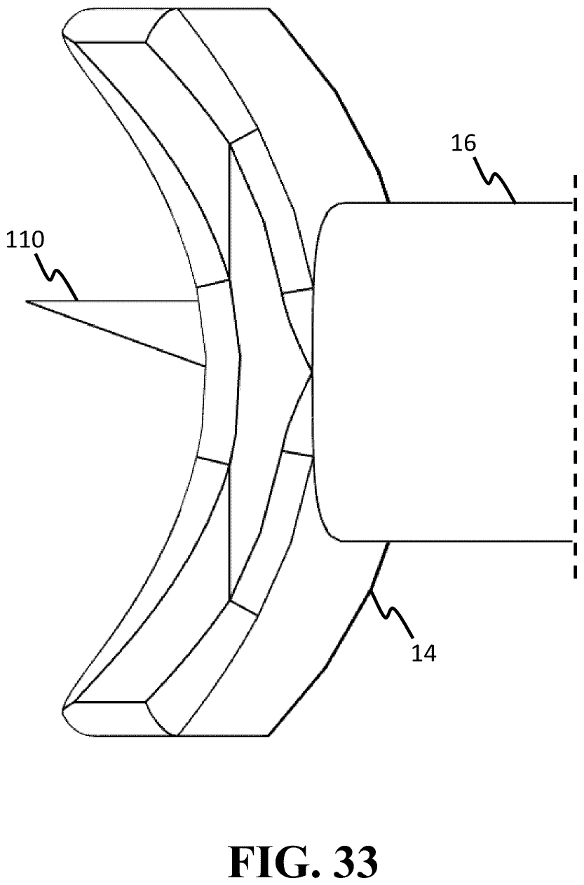

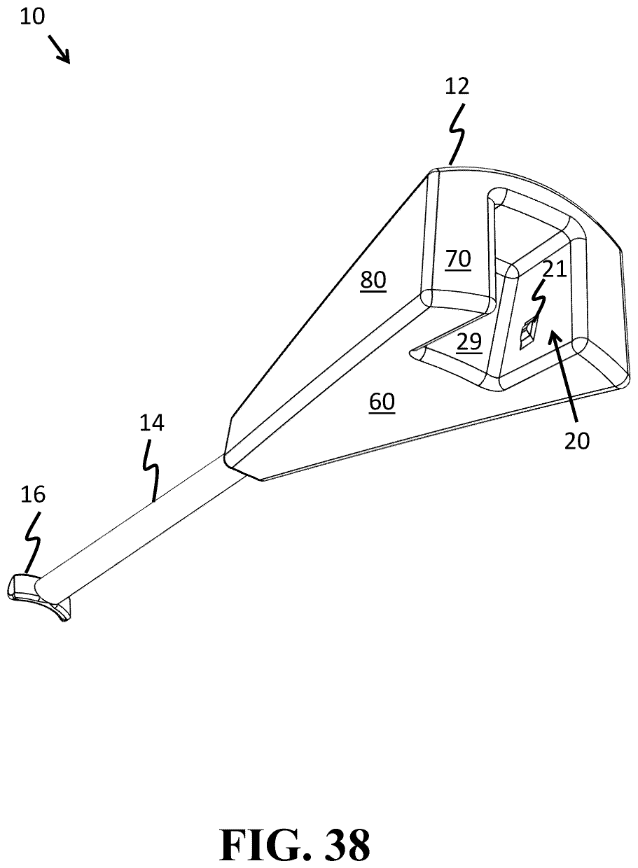

Referring to FIGS. 1-4, a shaft 14 extends distally from handle 12, according to some embodiments of the present disclosure. Shaft 14 may include a member extending from handle 12 to accommodate passage of an instrument (such as an instrument 100 as described in greater detail below) through the shaft 14. In some embodiments, shaft 14 is a tube-shaped member having a curvilinear profile. In other embodiments, and as described in greater detail below with reference to FIGS. 18-40, the shaft 14 includes a straight tube-shaped member. Of course, depending on the implementation, the shaft 14 may take any other suitable shape providing an elongated protrusion distal to handle 12 in order to provide the advantages discussed herein. In some embodiments, shaft 14 includes a length of about forty-five millimeters. Shaft 14 may be comprised of a rigid material sufficient to maintain its shape when a resilient instrument, such as a metal needle, is passed through channel 24. In some embodiments, shaft 14 is integrally formed with handle 12 in a one-piece construction. In other embodiments, shaft 14 comprises a separate component mechanically attached to handle 12 at a joint between shaft 14 and handle 12.

Referring further to FIG. 4, a channel 24 is defined through handle 12 and at least partially through shaft 14. A port 22 may be open at the proximal end of channel 24. For instance, port 22 may be open to receiver 20 such that an instrument (e.g., the instrument 100) may be inserted through receiver 20 and into port 22 to pass the instrument 100 into channel 24 and at least partially through shaft 14. The port 22 includes a chamfered, or tapered, opening having a funnel shape in some embodiments to better facilitate insertion of an instrument into channel 24 without damaging the instrument 100. Channel 24 may extend through shaft 24 toward the distal end of shaft 14. In some embodiments, an opening 18 is defined in shaft 14 at a location between foot 16 and handle 12. Thus, the opening 18 may be open to channel 24 such that the instrument 100 inserted through channel 24 may exit through the opening 18 and project distally from shaft 14. In some embodiments, opening 18 is about ten millimeters from foot 16. As shown in FIG. 4, in some embodiments, channel 24 includes an exit angle 30 such that the instrument will project out of channel 24 distal to foot 16. The exit angle of opening 18 in some embodiments is between about zero degrees and about sixty degrees relative to the centerline longitudinal axis of channel 24.

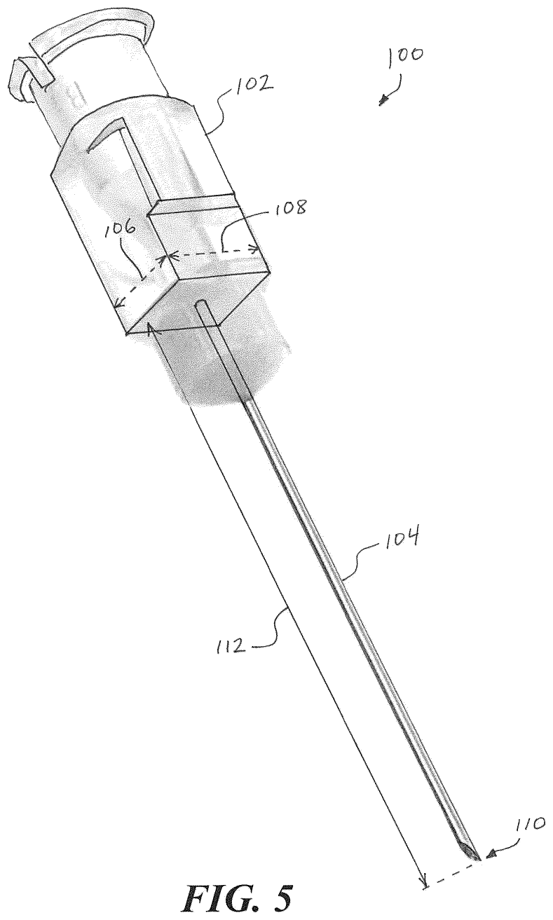

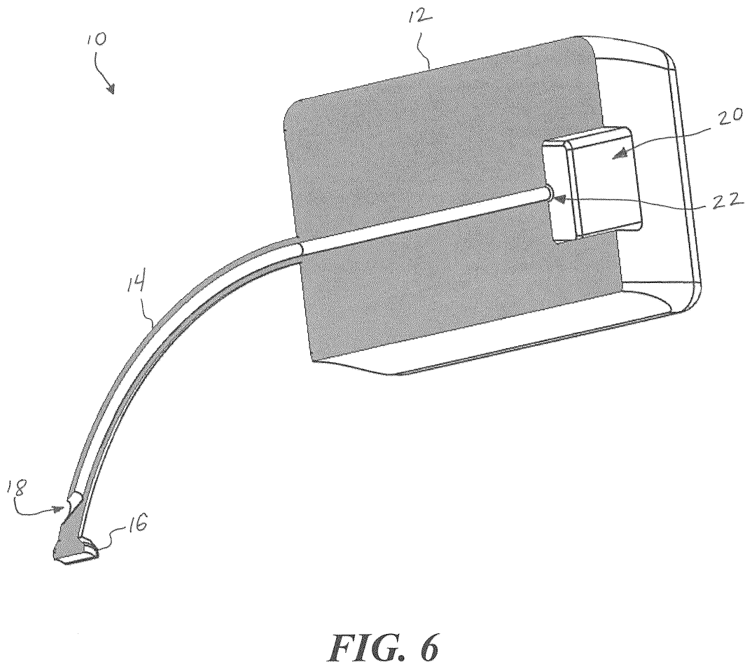

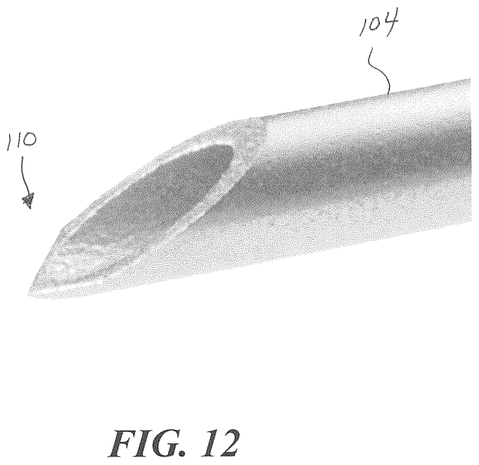

As mentioned above, the shaft 14 may be configured to accommodate passage of the instrument 100 therethrough. Referring to FIGS. 5 and 6, in some embodiments, the instrument 100 includes a needle having a hub 102 and a tube 104 (e.g., needle tube, a rod, a needle rod, etc.). In various other embodiments, instrument 100 may be any suitable instrument to be passed through channel 24 and project from shaft 14 to make an incision in tissue. In embodiments where instrument 100 is a needle, the instrument includes a tube length 112 and a tip 110 (e.g., a needle tip, a sharp end, a cutting tip, etc.) at the distal end of the tube 104. In some embodiments, needle of the instrument 100 includes an 18-gauge spinal needle configured with a Quincke beveled tip, as shown in FIG. 12.

Depending on the implementation, hub 102 may include a rectangular profile with a hub width 106 and a hub height 108. In some embodiments, hub 102 includes a portion having a shape and dimension corresponding to the shape and dimension of receiver 20 such that a portion of hub 102 may be received axially into receiver 20, thereby angularly securing hub 102 in handle 12 such that the tube 104 does not rotate angularly when hub 102 is received in receiver 20. Thus, hub 102 may be inserted axially into receiver 20 and may be held in place by a user during a procedure.

Due to the resiliency of the tube 104, instrument 100 may exhibit a tendency in some applications to be biased axially away from handle. A user may overcome this bias by maintaining pressure against the hub 102 into receiver 20 to keep the hub fully seated in receiver 20. During a procedure, a user may release the pressure from hub 102 to automatically withdraw tip 110 back into opening 18. As such, the fitment between hub 102 and receiver 20 provides a safety mechanism allowing a surgeon to quickly and easily retract tip 110 by releasing the pressure against hub 102.

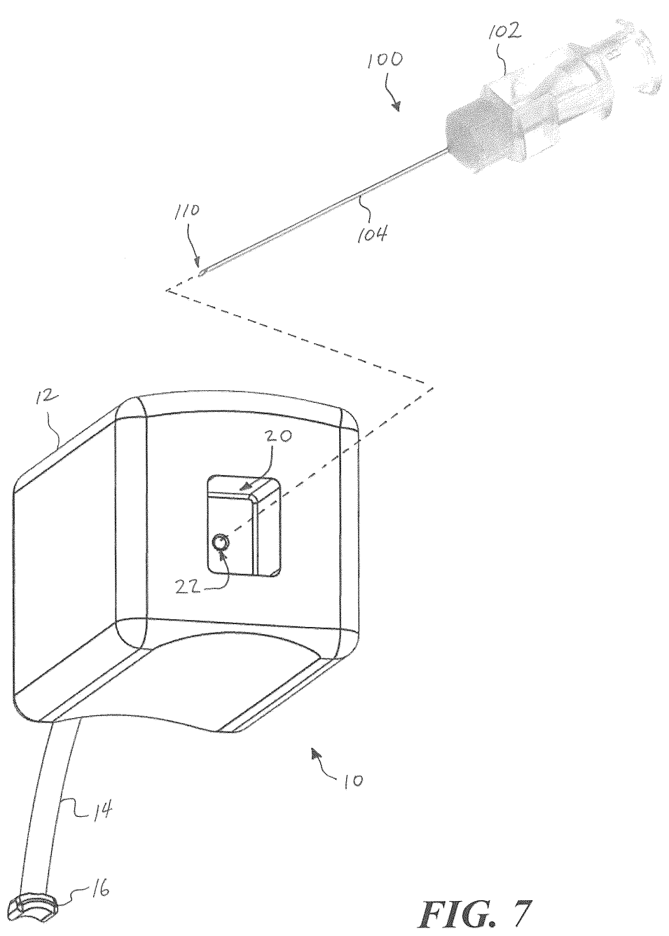

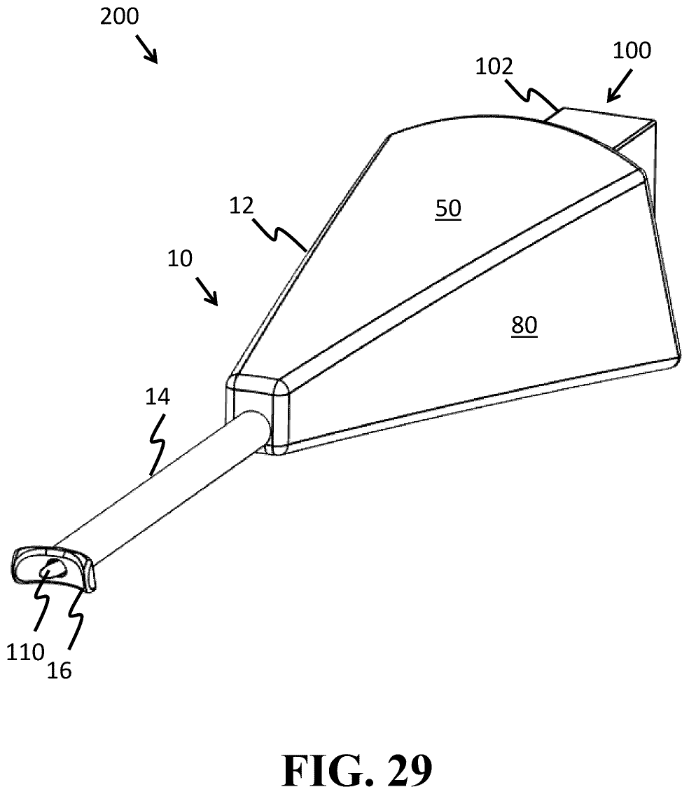

Referring to FIG. 7, during use, the instrument 100 may be inserted into apparatus 10 by inserting tip 110 into port 22 and advancing tube 104 through the channel 24 defined inside handle 12 and shaft 14 until hub 102 is received and fully seated in receiver 20. As shown with additional reference to FIGS. 29-31, an apparatus 200 may be provided. The apparatus 200 may include the apparatus 10 as discussed herein, with the instrument 100 assembled thereto as discussed herein.

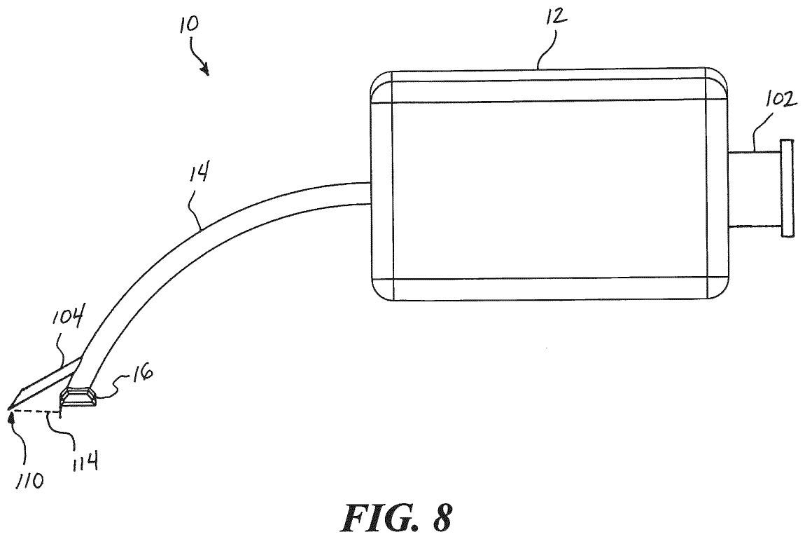

In some embodiments, the tube length 112 may be dimensioned such that when hub 102 bottoms out in receiver 20, tip 110 protrudes distally from shaft 14, as shown in FIG. 8. For example, tube length 112 may be between about eight and nine centimeters. In further embodiments, tube length 112 is about 8.9 centimeters. As such, the receiver recess depth 54 provides an axial stop for hub 102 during insertion. By precisely optimizing receiver shaft 14 length, opening 18 location, handle 12 length, receiver recess depth 54 and needle length 112, the apparatus 10 provides a desired end effector profile, as shown in FIG. 8. For example, tip 110 may extend distal to the leading edge of foot 16 by a projection distance 114 of about one to ten millimeters when hub 102 is fully seated in receiver 20. In some embodiments, projection distance 114 is between about one to two millimeters. In other embodiments, projection distance 114 is variable to achieve optimal performance of apparatus 10.

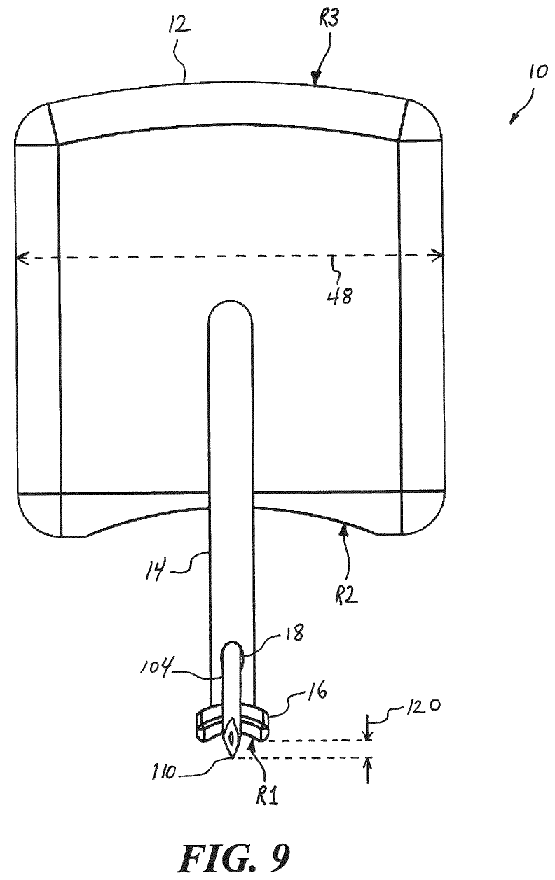

Referring to FIG. 9, the cutting depth 120 that tip 110 extends below foot 16 may also be optimized by controlling the dimensions of shaft 14 length, opening 18 location, tube 112 length, handle 12 length, and receiver recess depth 54. For example, when hub 102 is fully seated in receiver 20, tip 110 may extend a cutting depth 120 of zero such that tip 110 does not extend below foot 16. In other embodiments, tip 110 extends a cutting depth 120 of between about 0.5 millimeters and about three millimeters. Cutting depth 120 is variable in some embodiments and may be adjusted to achieve optimal performance of the apparatus 10.

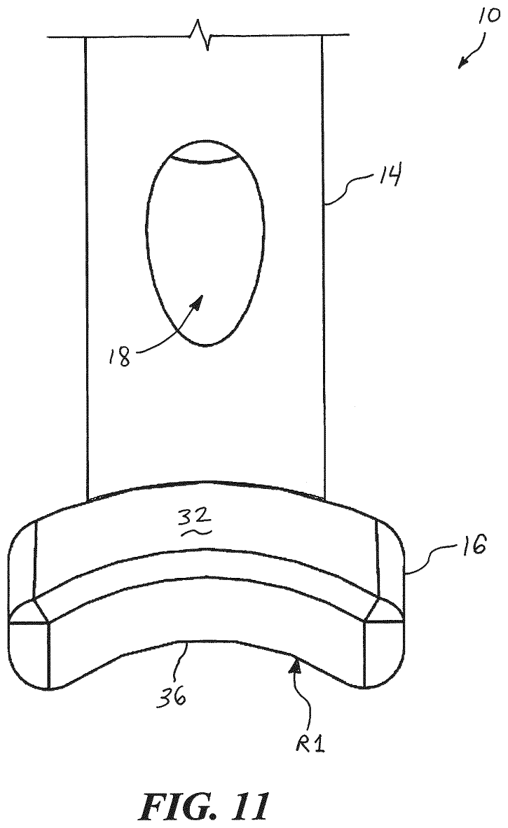

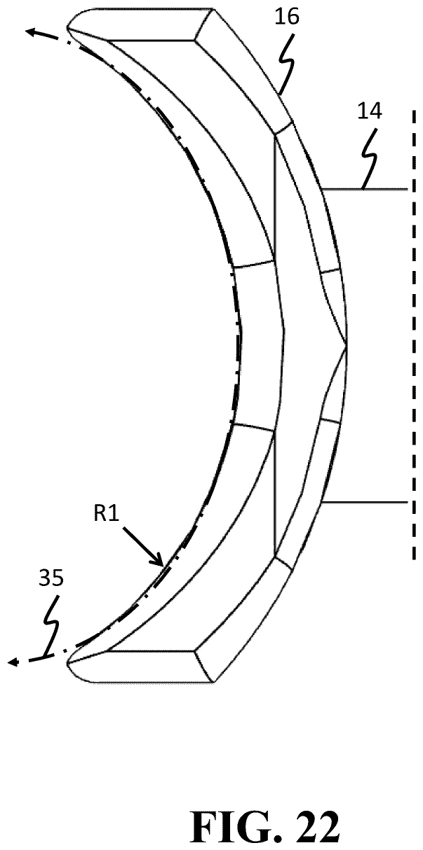

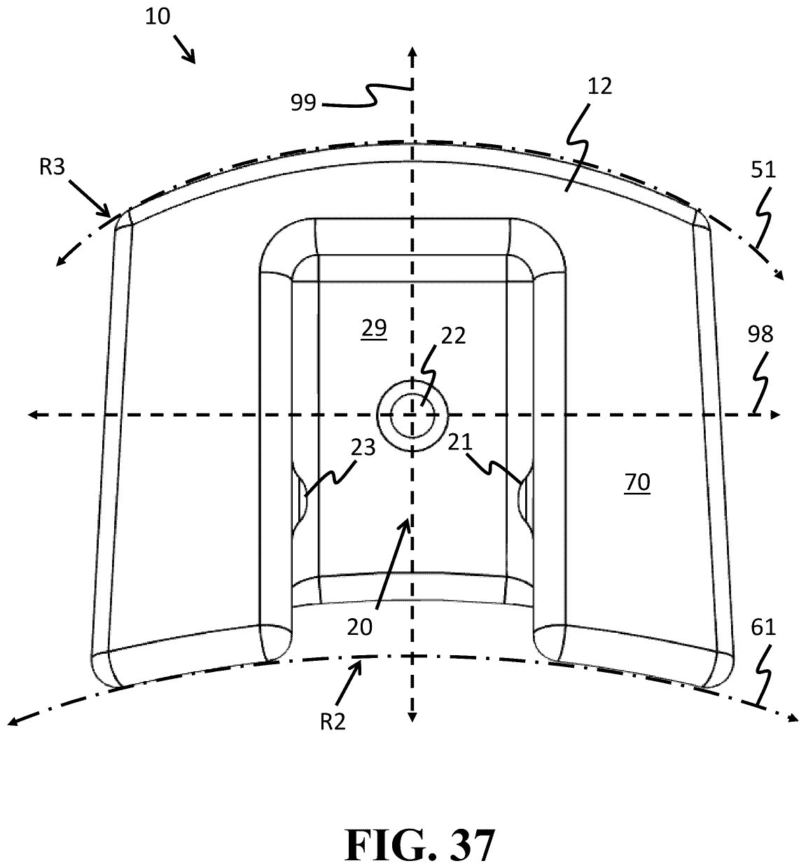

Referring further to FIGS. 9 and 11, in some embodiments, foot 16 includes a concave contact surface 36 including a first radius of curvature R1. For example, and as shown with additional reference to FIG. 22, the concave contact surface 36 may form a contact surface plane 35 that defines the first radius of curvature R1. In some embodiments, first radius of curvature R1 is dimensioned to approximate the surface contour of a flexor tendon sheath, thereby allowing foot 16 to slide along the surface of the flexor tendon sheath during a procedure. In some embodiments, bottom surface 60 of handle 12 includes a second radius of curvature R2, and top surface 50 of handle 12 includes a third radius of curvature R3. For example, and as shown with additional reference to FIG. 37, the top surface 50 may form a top surface plane 51 that defines the second radius of curvature R2, and the bottom surface 60 may form a bottom surface plane 61 that defines the third radius of curvature R3. Depending on the implementation, some or all of each such curved surface (e.g., the concave contact surface 36, the bottom surface 60, and/or the top surface 50) includes a thirty-degree arc.

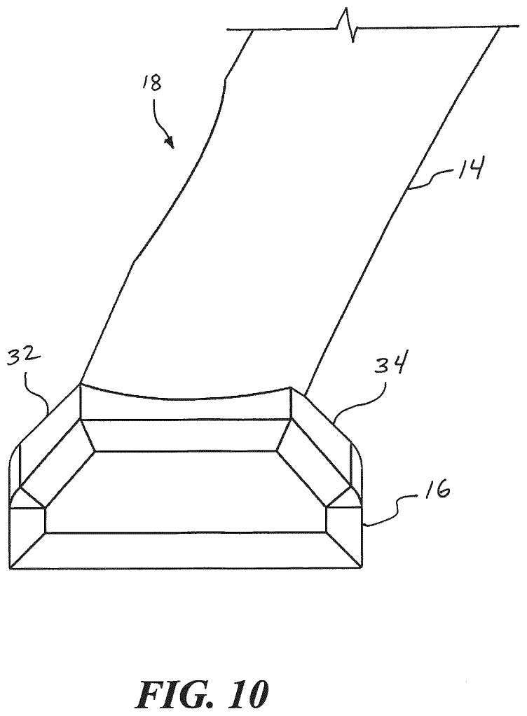

Referring to FIG. 10, in some embodiments, foot 16 includes a leading edge ramp 32 and a trailing edge ramp 34. Each ramp 32, 34 may provide an inclined surface that operates to lift tissue off the tendon sheath as the foot 16 travels relative to the tendon sheath, thereby providing enhanced access to the A1 pulley with the tip 110 to perform a dissection of the pulley. Each ramp 32, 34 may include a flat inclined profile, or a curved inclined profile, in some embodiments.

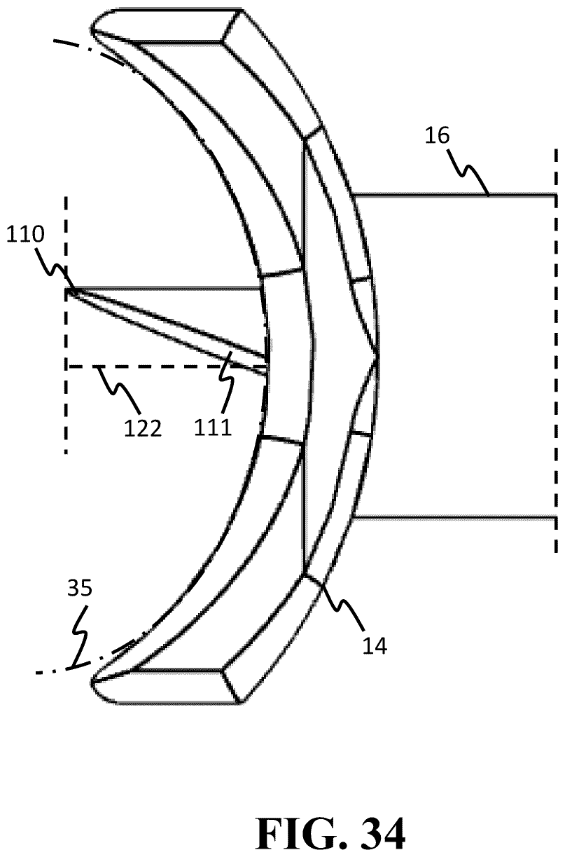

Referring to FIG. 12, an embodiment of the tip 110 at the distal end of a tube 104 is shown. Tip 110 may include a sharp point that may be used to cut tissue by moving the needle tip forward and backwards or from side to side to dissect tissue. In other words, the tip 110 may be configured to dissect tissue during a surgical procedure. In some embodiments, and as shown with additional reference to FIG. 34, the tip 110 includes a beveled surface 111. For example, the tip 110 may include a Quincke beveled needle tip. During use, the tip 110 may be oriented with the beveled surface 111 facing upwardly as shown in FIG. 12. In other applications, the beveled surface 111 of tip 110 may be oriented facing down, to either side, or rotated angularly to either side. Accordingly, the apparatus 10 may provide the ability to angularly lock the beveled surface 111 at a desired orientation by engaging the hub 102 in the receiver 20 to prevent rotation of the instrument 100 during use.

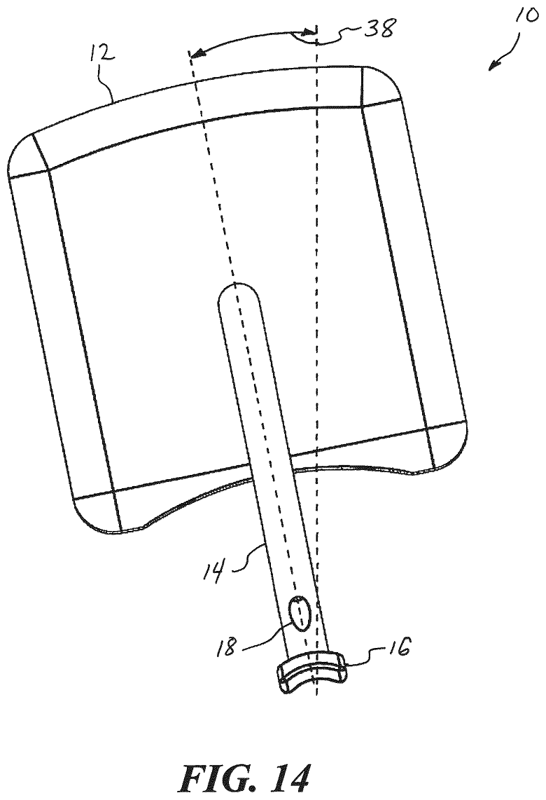

Referring to FIG. 13, a cross-sectional view of an embodiment of the apparatus 10 is shown, according to some embodiments. As mentioned above, the apparatus 10 may include the shaft 14 (in a curved configuration as shown) and the channel 24 extending through the apparatus 10 from the receiver 20 to the opening 18. As shown, the channel 24 may be dimensioned to accommodate the outer diameter of the tube 104 of the instrument 100 (e.g., an 18-gauge spinal needle). In some embodiments, foot 16 extends an offset distance 56 below the bottom surface of handle 12. As such, angular rotation of apparatus 10 away from a vertical plane, as shown in FIG. 14, by an angle 38 allows a user to readily perceive that the apparatus 10 has pivoted away from the vertical plane. This configuration aids a surgeon in ensuring concise control and proper alignment of the apparatus 10 during a procedure.

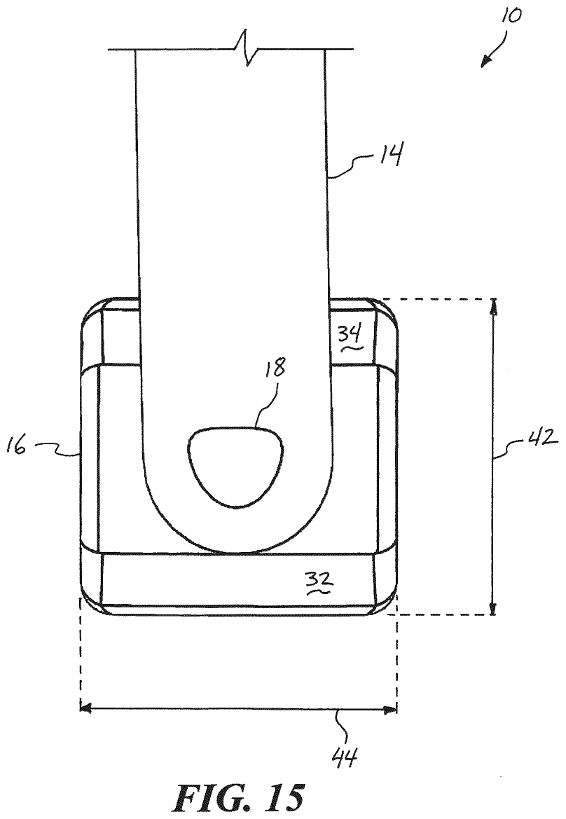

Referring to FIG. 15, in some embodiments, foot 16 includes a square profile including a foot length 42 and a foot width 44 being the same. In some embodiments, foot length 42 and foot width 44 are about five millimeters. In further embodiments, foot length 42 and foot width 44 may be dimensioned to any suitable sizes for performing a desired operation.

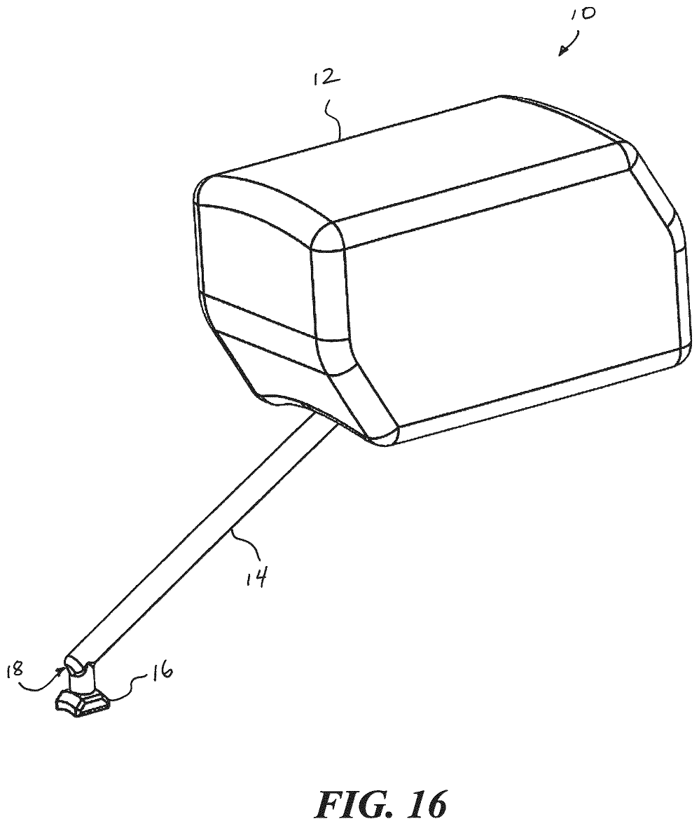

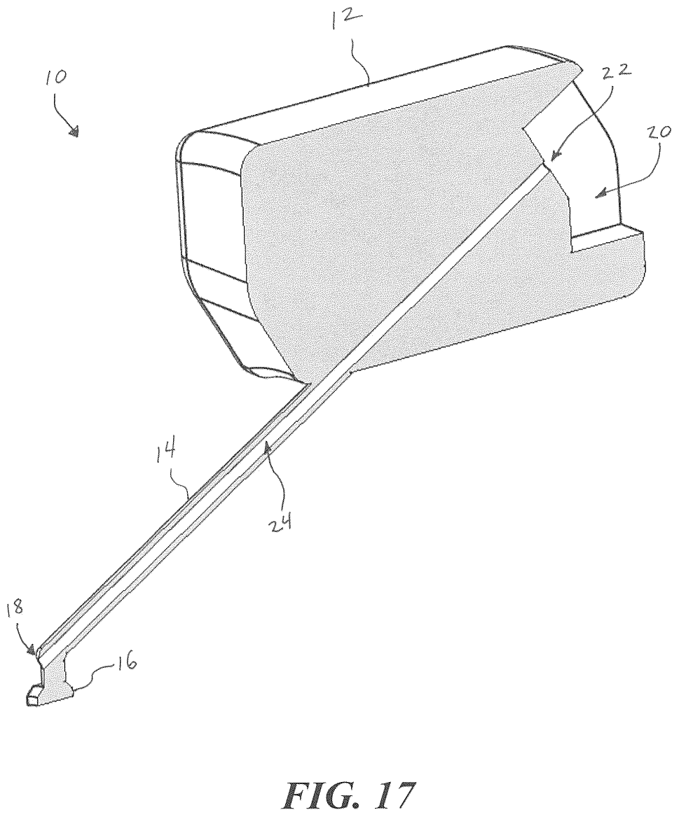

Referring to FIG. 16 and FIG. 17, in some embodiments, apparatus 10 includes a handle 12 and a straight shaft 14. In such embodiments, shaft 14 includes a straight channel 24 extending through handle 12 and shaft 14 from port 22 to opening 18. In such embodiments, when an instrument is inserted into receiver 20, the tube 104 extends distally from opening 18 distal to foot 16.

In some embodiments, apparatus 10 includes any suitable rigid material for guiding an instrument during surgery. In some embodiments, apparatus 10 includes a polymer material. As such, a surgical procedure utilizing real-time ultrasound imaging during the procedure may readily identify the precise location of the metal needle tip due to the imaging contrast between the metal needle material and the polymer material of the apparatus 10.

In further embodiments, the present disclosure provides a method comprising the steps of (a) providing a surgical tool including a handle and a shaft with a channel defined through the handle and at least partially through the shaft; (b) inserting an instrument through the channel such that a portion of the instrument extends distally from the shaft; and (c) manipulating the tool such that the portion of the instrument extending distally from the shaft engages and dissects the tissue. In some embodiments, the tissue includes an A1 pulley in patient's hand.

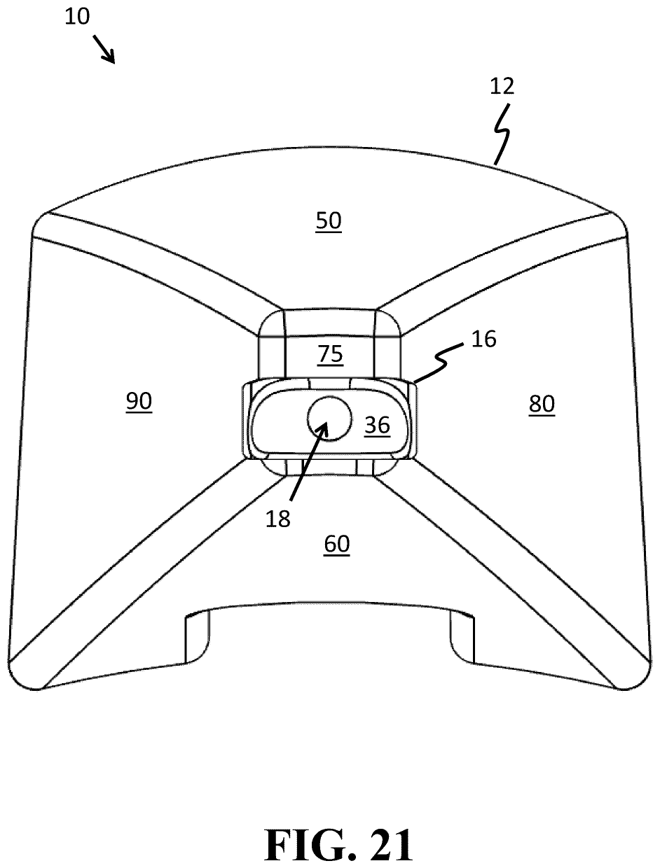

Referring generally to FIGS. 18-46, the apparatus 10 is shown, according to further embodiments of the present disclosure. Depending on the implementation, the apparatus 10 as described with reference to FIGS. 18-46 may include some or all of the components of the apparatus 10 as discussed above, in the same or in varied configurations. For instance, as suggested above, and as shown with reference to FIGS. 18-25, the apparatus 10 may include a body having the handle 12 and the shaft 14 extending distally from the handle 12. Similarly, the receiver 20 may be defined in a proximal end of the handle 12 opposite the shaft 14, the receiver 20 may include the port 22; the foot 16 may be disposed on a distal end of the shaft 14 opposite the handle 12; and the channel 24 may be defined from the port 22 to the opening 18. As shown with particular reference to FIG. 21, the handle 12 may include the top surface 50, the bottom surface 60, a left surface 80, and a right surface 90. The top, bottom, left, and right surfaces 50, 60, 80, and 90 may narrow in width from a rear surface 70 (on the proximal end of the handle 12) to a front surface 75 (on the distal end of the handle 12), from which the shaft 14 may project.

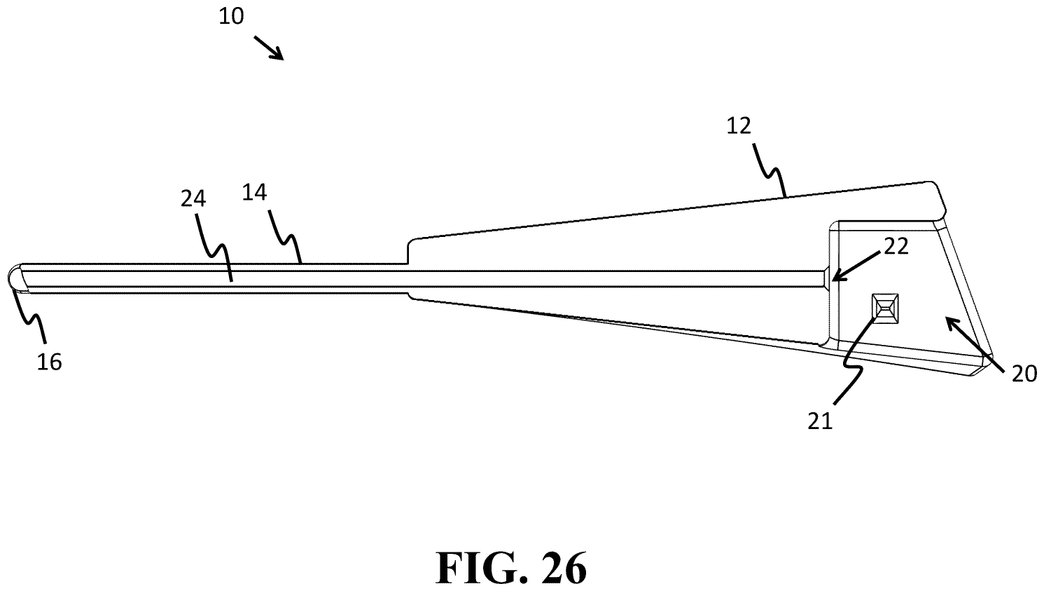

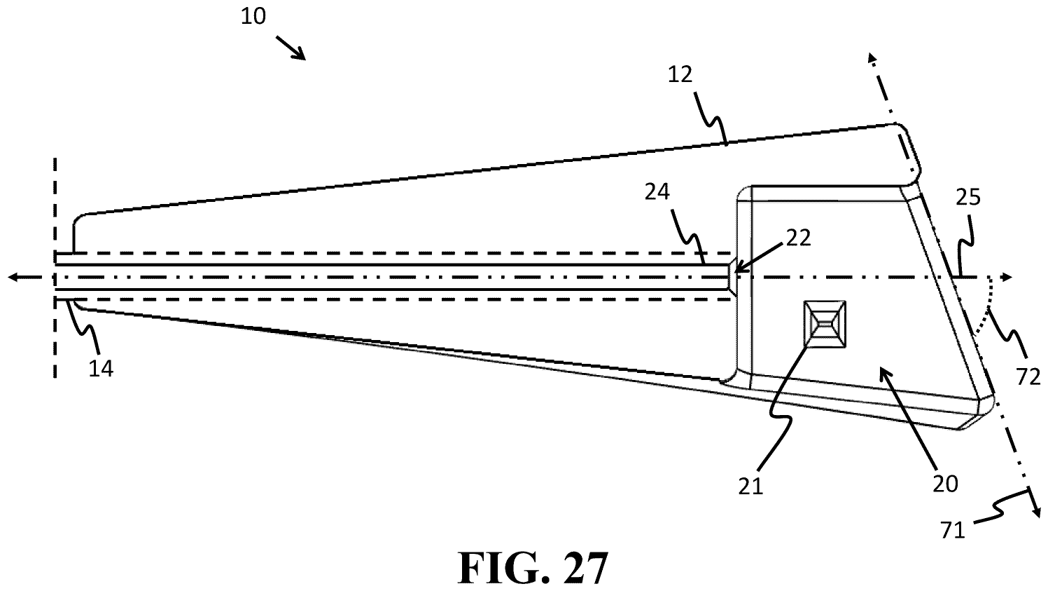

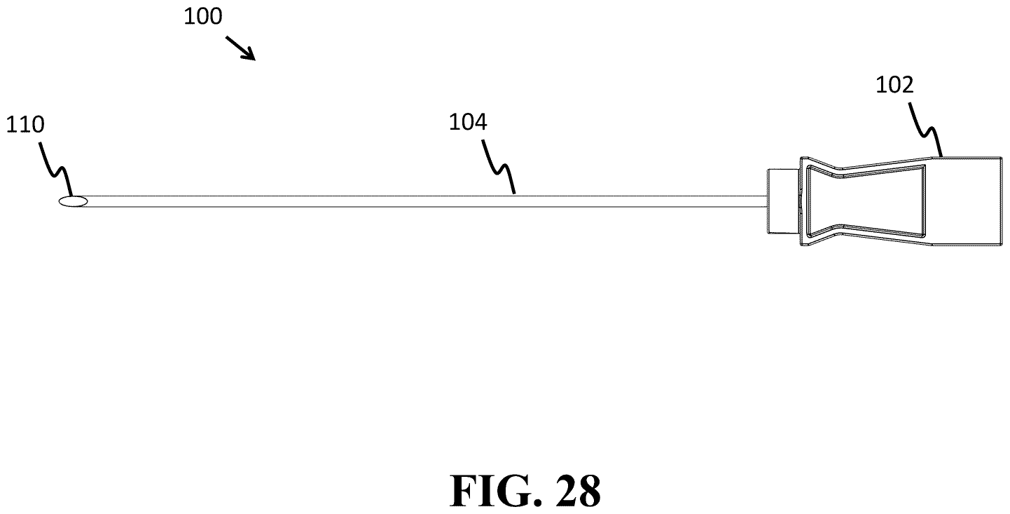

Referring to FIGS. 26 and 27, and as mentioned above with reference to FIGS. 1-4, the shaft 14 may include a straight tube-shaped member. Thus, the channel 24 defined through the handle 12 and the shaft 14 may be straight (or substantially straight). In this sense, and as shown with particular reference to FIG. 27, the channel 24 may define a longitudinal axis 25 that is straight (or substantially straight) and extends from the port 22 to the opening 18. Of course, as mentioned above with reference to FIGS. 5 and 6, the instrument 100 (including the needle having the tube 104, the tube length 112, and the tip 110) may be axially received by the apparatus 10, such that the instrument 100 is configured to be passed through the channel 24 and project from the shaft 14 to make an incision in tissue. Thus, the channel 24 may be configured to receive the cutting instrument 100 including the tip 110 that is configured to dissect tissue during a surgical procedure. In embodiments where the shaft 14 includes a straight tube-shaped member, the tube 104 of the instrument 100 may be straight, as shown with reference to FIG. 28.

As mentioned above with reference to FIGS. 1-4, in some embodiments, the shaft 14 is integrally formed with the handle 12 in a one-piece construction, while in other embodiments the shaft 14 is a separate component mechanically attached to handle 12. Referring further to FIG. 27, the shaft 14 may be a separate component that extends through the handle 12 (e.g., from the port 22 on an inner face 29 of the receiver 20 (shown with reference to FIG. 37) to the front surface 75 of the handle 12 (shown with reference to FIG. 21)), and projects from the handle 12 (e.g., from the front surface 75 of the handle 12). In further embodiments, the apparatus 10 may not include the shaft 14. For instance, the foot 16 may simply be disposed on or otherwise connected to a distal end of the handle 12. In any case, the channel 24 may be defined from the port 22 to the opening 18 of the foot 16.

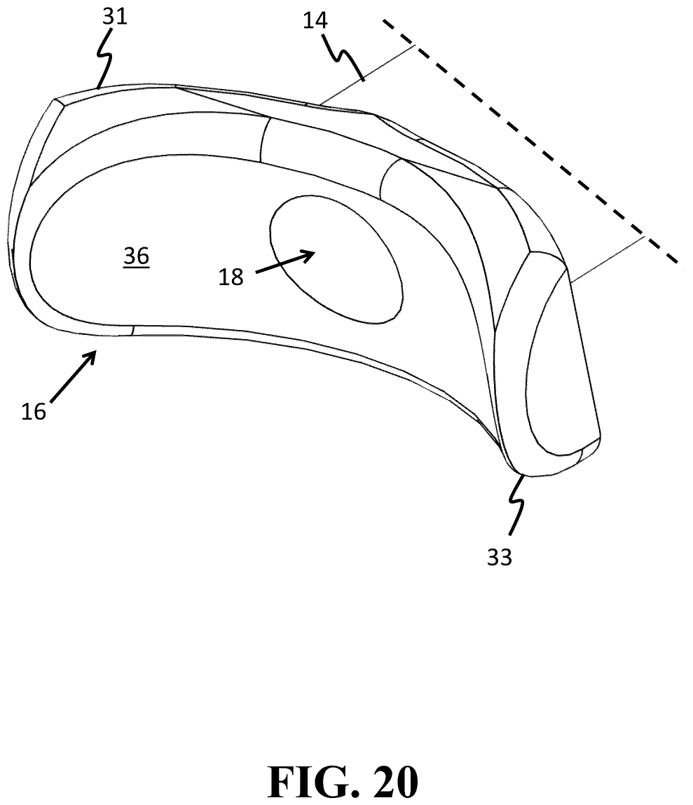

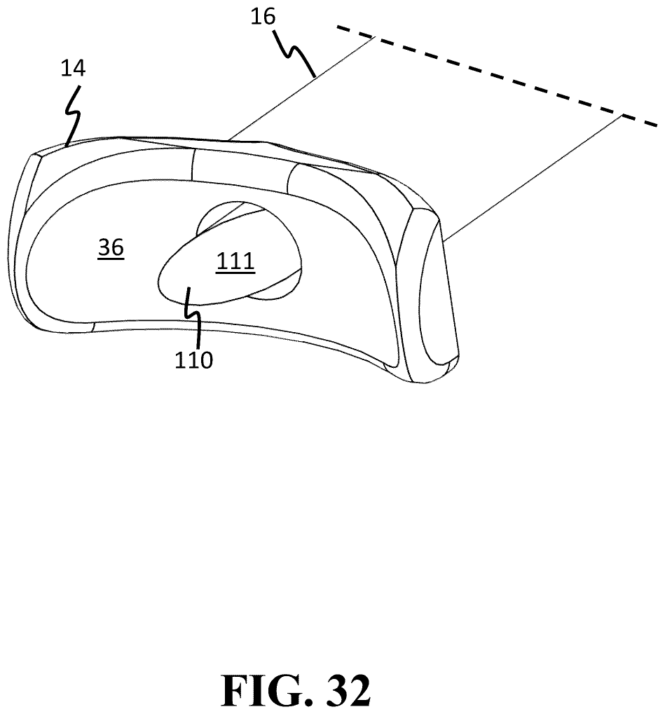

As mentioned above with reference to FIG. 8, the tube length 112 may be dimensioned such that when hub 102 bottoms out (or is fully seated) in receiver 20, tip 110 protrudes distally from shaft 14. Thus, the cutting instrument 100 may include the hub 102 and the tube 104 that is received by the channel 104, such that when the hub 102 is seated within the receiver 20, the tip 110 of the cutting instrument 100 projects distally out of the opening 18. As further discussed above, the tube length 112 may be configured such that the tip 110 extends distal to the leading edge of the foot 16 by the projection distance 114. However, in other embodiments where the tube 104 is straight, when the hub 102 of the instrument 100 is received axially into the receiver 20, the tip 110 may exit from a central location on the concave contact surface 36 of the foot 16, as shown with reference to FIGS. 29-34. In this sense, and as shown with reference to FIGS. 20 and 32, the opening 18 may be positioned directly on the concave contact surface 36 (rather than on the shaft 14 between the foot 16 and the handle 12 as discussed above). Thus, in such embodiments where the foot 16 includes the opening 18 thereon, the foot 16 may include the concave contact surface 36, and the opening 18 may be disposed on the concave contact surface 36. Accordingly, the tip 110 of the cutting instrument 100 may be configured to project distally out of the opening 18 of the foot 16.

As mentioned above with reference to FIG. 9, the cutting depth 120 that the tip 110 extends below foot 16 may also be optimized by controlling the dimensions of the shaft 14 length, the opening 18 location, the tube 112 length, the handle 12 length, and the receiver recess depth 54. Similarly, and as shown with reference to FIG. 34, in such embodiments where the opening 18 is positioned directly on the concave contact surface 36 (rather than on the shaft 14), when the hub 102 of the instrument 100 is received axially into the receiver 20, the tip 110 may achieve a cutting clearance 122 relative to the foot 16. For example, the cutting clearance 122 may be defined as the maximum distance (measured along the longitudinal axis 25) that the tip 110 clears the concave contact surface 36 of the foot 16 (or, as shown with reference to FIGS. 22 and 34, the contact surface plane 35 formed thereby). As discussed in greater detail below, the foot 16 may include opposing lateral ends that extend distally relative to the opening 18. The cutting clearance 122 may be configured such that when the hub 102 is seated within the receiver 20, the tip 110 of the cutting instrument 100 projects distally out of the foot 16 and is prevented from extending further from the opening 18 than the opposing lateral ends of the foot 16.

In some embodiments, the cutting clearance 122 may be between about one and about two millimeters. In some embodiments, the cutting clearance 122 is between about 1.4 and about 1.6 millimeters. In further embodiments, the cutting clearance 122 is about 1.5 millimeters. Depending on the implementation, the cutting clearance 122 may be adjusted (whether by providing a longer tube 104, a shorter shaft 14, or some other dimensional variation) to achieve optimal performance of the apparatus 10. Advantageously, the cutting clearance 122 may be configured such that the tip 110 is only allowed to extend so far as to sever the A1 pulley without damaging the underlying tendon, as discussed herein, without causing inadvertent damage to the hand.

As mentioned above with reference to FIG. 12, the tip 110 may be oriented with the beveled surface 111 facing upwardly. In other embodiments, and with further reference to FIG. 34, the beveled surface 111 of the tip 110 may be oriented to the side. For example, as shown with reference to FIG. 37, the apparatus 10 may form a reference x-axis 98 and a reference y-axis 99 extending from longitudinal axis 25 (which may act as a reference z-axis). As shown with reference to FIG. 34, the beveled surface 111 of the tip 110 may be oriented perpendicular to the longitudinal axis 25 and parallel to the reference x-axis 98, such that the beveled surface 111 faces to either lateral side of the apparatus 10. Such a configuration may orient the tip 110 in an upright cutting orientation transverse to the tendon sheath, similar to a knife blade.

As mentioned above with reference to FIG. 28, the tube 104 may be straight. Thus, the instrument 100 may be (or otherwise include) an off-the-shelf product. For example, the needle of the instrument 100 may be an off-the-shelf spinal needle. In particular, the off-the-shelf spinal needle may be a Mckesson spinal needle, sized at eighteen-gauge and three and one-half inches, or a similar off-the-shelf product. Of course, the aforementioned embodiment is merely exemplary in nature, and it should be appreciated that any suitable instrument 100 (and needle tube 102 and hub 104 therein) may be used with the apparatus 10 in order to achieve the advantages discussed herein.

As suggested above, when the foot 16 is positioned on the tendon sheath, the concave contact surface 36 aids in centering the foot 16 on the tendon sheath and sweeping soft tissue off the tendon sheath. In this sense, the concave contact surface 36 may be shaped to accommodate the tendon sheath. For instance, and as shown with reference to FIG. 20, the concave contact surface 36 may include opposing lateral ends that extend distally relative to the opening 18. For example, the concave contact surface 36 may form a first lip 31 on one end of the foot 16, and a second lip 33 on another end of the foot 16 opposite the first lip 31. Each of the first and second lips 31, 33 may sit on opposing sides of the tendon sheath, and each may sweep soft tissue from such opposing sides from the surface of the tendon sheath.

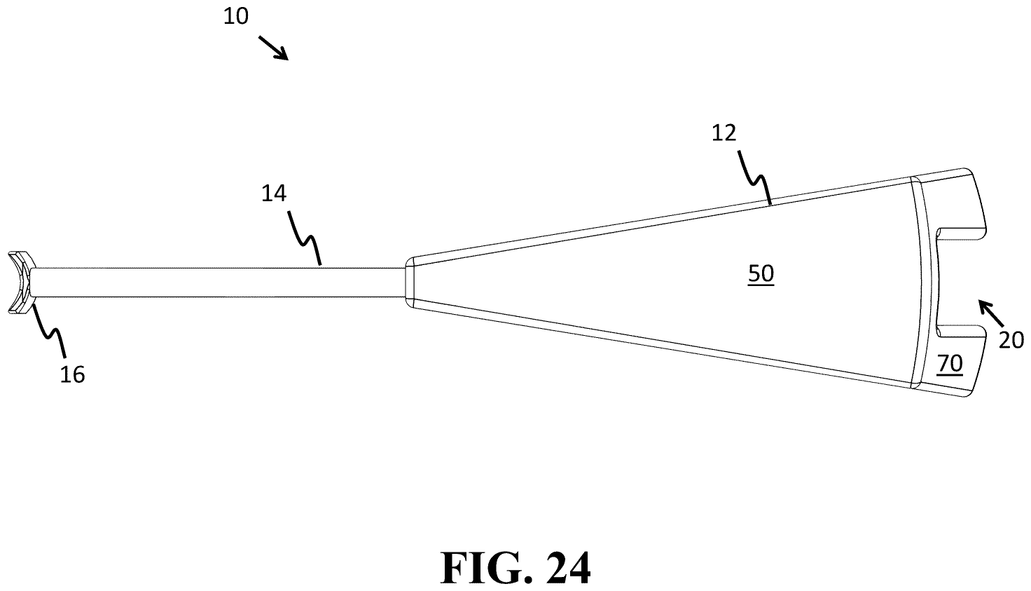

As mentioned above with reference to FIGS. 9 and 11, and as shown with reference to FIG. 22, the foot 16 may include the concave contact surface 36 which may form the curvilinear contact surface plane 35 that defines the first radius of curvature R1, which may be dimensioned to approximate the surface contour of a flexor tendon sheath. In some embodiments, and as shown with reference to FIGS. 18 and 24, the handle 12 may include the rear surface 70. As discussed in greater detail below, the concave contact surface 36 and the rear surface 70 of the handle 12 may have similar curvature.

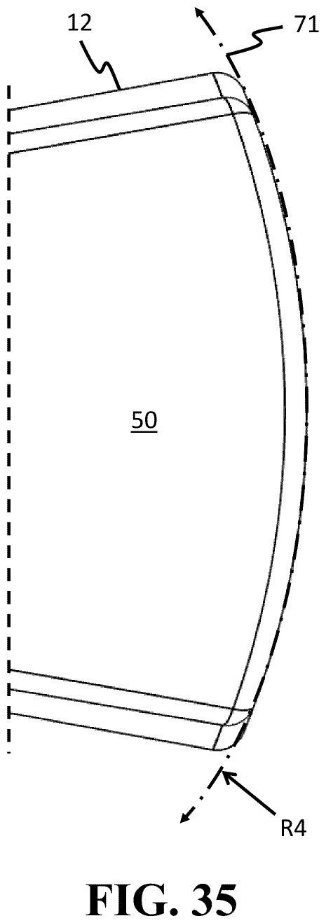

As shown with reference to FIG. 35, the rear surface 70 may define a rear surface plane 71. The rear surface plane 71 formed by the rear surface 70 may define a fourth radius of curvature R4. In some embodiments, each of R1 and R4 are equivalent (or substantially equivalent). For example, each of R1 and R4 may form about a thirty-degree arc. In this sense, when the foot 16 is positioned on the tendon sheath as discussed above, the equivalent arcs formed by the concave contact surface plane 35 and the rear surface plane 71 (forming equivalent radii of curvature R1, R4, respectively), may allow a user to readily perceive the orientation of the channel 24, and thus the tip 110. This configuration may further aid a surgeon in ensuring concise control and proper alignment of the apparatus 10 during a procedure.

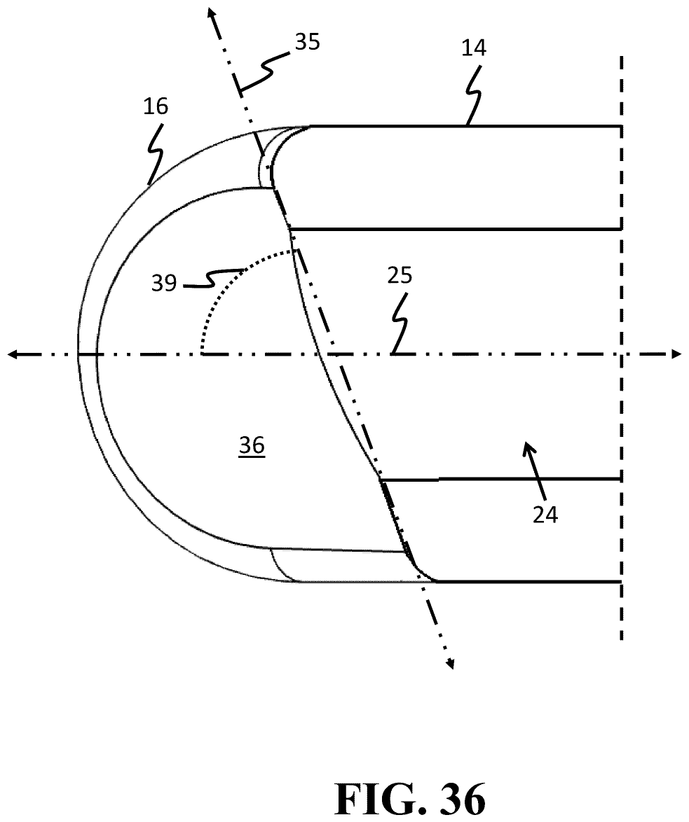

As shown with reference to FIG. 27, the aforementioned rear surface plane 71 may form a rear surface angle 72 relative to the longitudinal axis 25 of the channel 24 (or, more generally, relative to the shaft 14). On an opposite end of the apparatus 10, and as shown with reference to FIG. 36, the contact surface plane 35 formed by the concave contact surface 36 of the foot 16 may form a contact surface angle 39 relative to the longitudinal axis 25. In some embodiments, the rear surface angle 72 and the contact surface angle 39 are equivalent (or substantially equivalent). For example, each of the rear surface angle 72 and the contact surface angle 39 may be approximately seventy degrees. In other words, the rear surface 70 may be parallel to the concave contact surface 36. The equivalence of the rear surface angle 72 and the contact surface angle 39 may similarly aid a surgeon in ensuring concise control and proper alignment of the apparatus 10 during a procedure.

As mentioned above with reference to FIGS. 9 and 11, the bottom surface 60 of handle 12 may include the second radius of curvature R2, and the top surface 50 of handle 12 may include the third radius of curvature R3. For example, and as shown with reference to FIG. 37, the top surface 50 may form a curvilinear top surface plane 51 that defines the third radius of curvature R3, and the bottom surface 60 may form a curvilinear bottom surface plane 61 that defines the second radius of curvature R2. Each curved surface in some embodiments includes about a thirty-degree arc.

As mentioned above, with reference to FIG. 5, the instrument 100 may include a needle having the hub 102 and the tube 104. The hub 102 may include a portion having a shape and dimension corresponding to the shape and dimension of the receiver 20 such that a portion of the hub 102 may be received axially into the receiver 20. In some embodiments, and as shown with reference to FIGS. 27, 37, and 38, the receiver 20 includes a first detent 21 positioned on one side of the receiver recess width 26 (mentioned above with reference to FIG. 3), and a second detent 23 positioned opposite the first detent 21 on an opposite side of the recess width 26. The first and second detents 21, 23 within the receiver 20 may be configured to secure the hub 102 (and thus the instrument 100) when the hub 102 is received axially into the receiver 20. As mentioned above with reference to FIG. 7, the receiver recess depth 54 may provide an axial stop for hub 102 of the instrument 100 during insertion. As discussed in greater detail below, the first and second detents 21, 23 may similarly provide axial retention of the instrument 100 during insertion.

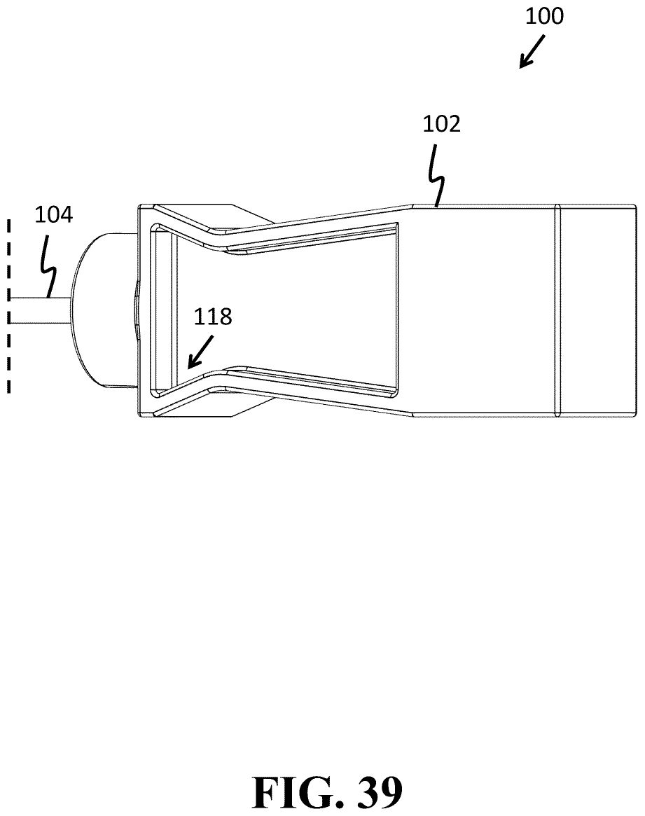

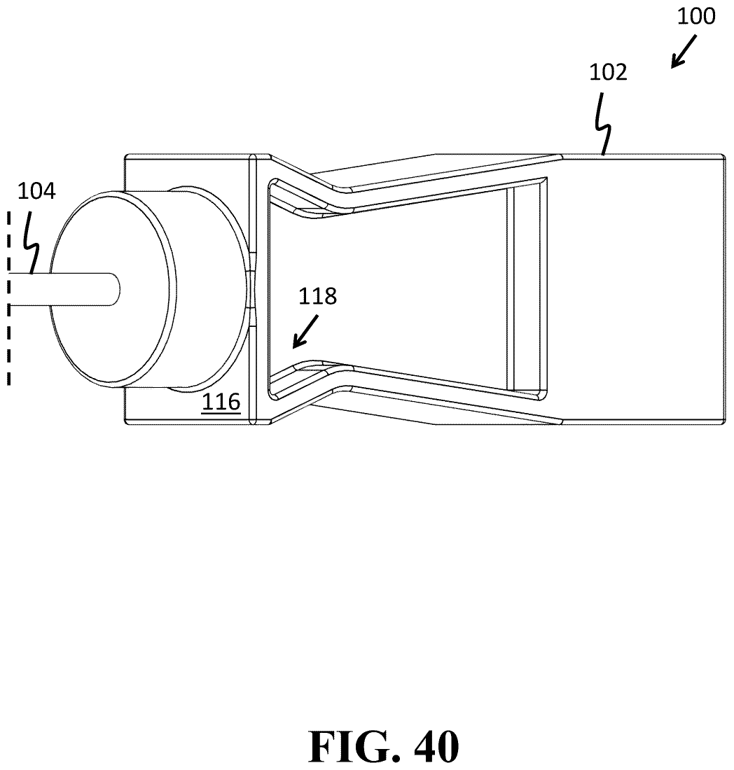

As shown with reference to FIGS. 39 and 40, the hub 102 may include a lip 116 and a recess 118. When the hub 102 is received axially into the receiver 20, the lip 116 may be pressed past the first and second detents 21, 23, until the hub 102 is seated against the inner face 29 of the receiver 20 (shown with reference to FIG. 37), at which point the first and second detents 21, 23 may be positioned within the recess 118. As such, the receiver recess depth 54 provides an axial stop for hub 102 during insertion. Accordingly, in order to withdraw the hub 102 from the receiver 20, the lip 116 may again need to be pressed past the first and second detents 21, 23. Thus, the engagement between the first and second detents 21, 23 of the receiver 20, and the lip 116 of the hub 102, may provide a sufficient amount of resistance against the hub 102 being axially withdrawn from the receiver 20, thus preventing inadvertent axial movements of the instrument 100 relative to the apparatus 10 once the hub 102 is axially inserted into the receiver 20.

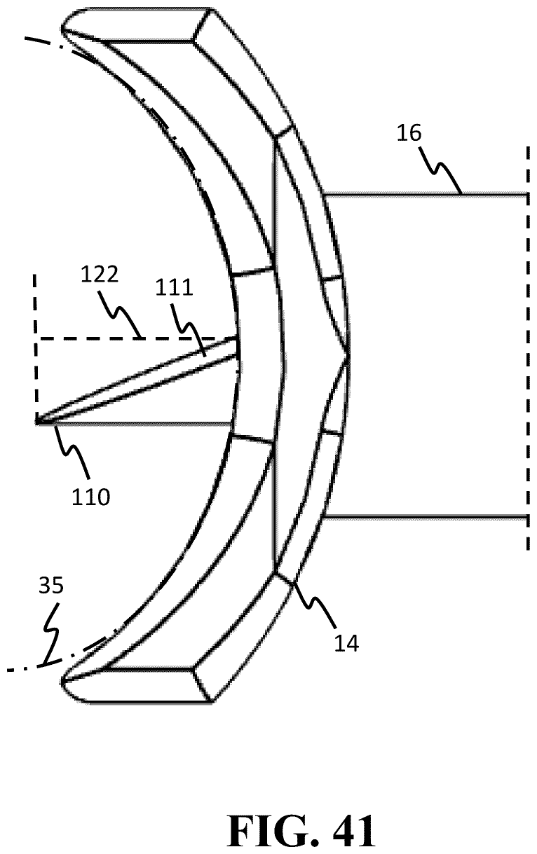

As mentioned above with reference to FIG. 34, the beveled surface 111 of the tip 110 on the instrument 100 may be oriented such that the beveled surface 111 faces to the side of the apparatus 10. The hub 102 (and, in general, the instrument 100) may be symmetrical along the longitudinal axis 25, such that the hub 102 may be axially received into the receiver 20 in a first orientation or a second orientation whereby the instrument 100 is rotated one-hundred and eighty degrees about the longitudinal axis 25. In this sense, when the hub 102 is axially received into the receiver 20 in the first orientation, the beveled surface 111 may face one side of the apparatus 10 as shown with reference to FIGS. 29 and 34), and when the hub 102 is axially received into the receiver 20 in the second orientation, the beveled surface 111 may face the opposite side of the apparatus 10 as shown with reference to FIG. 41.

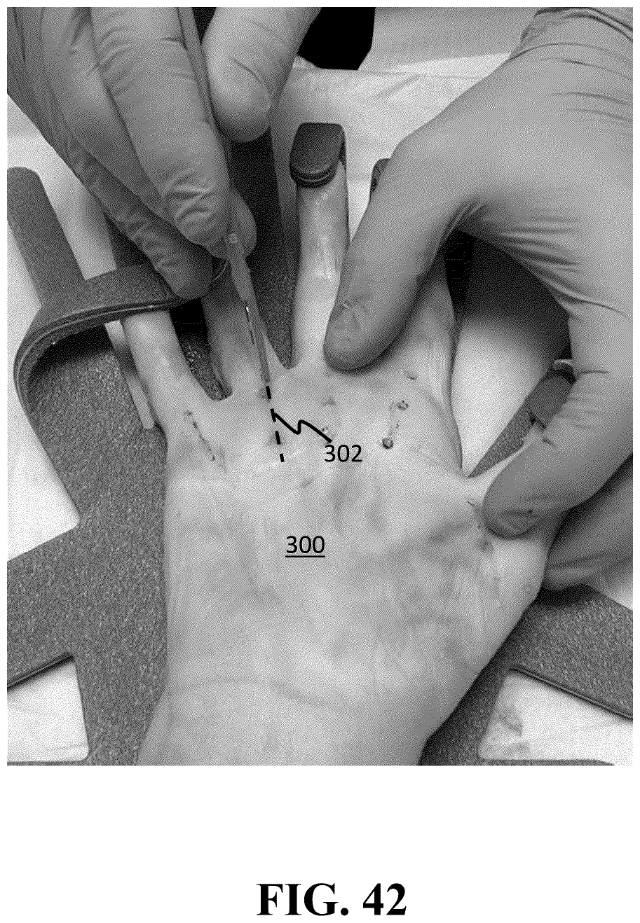

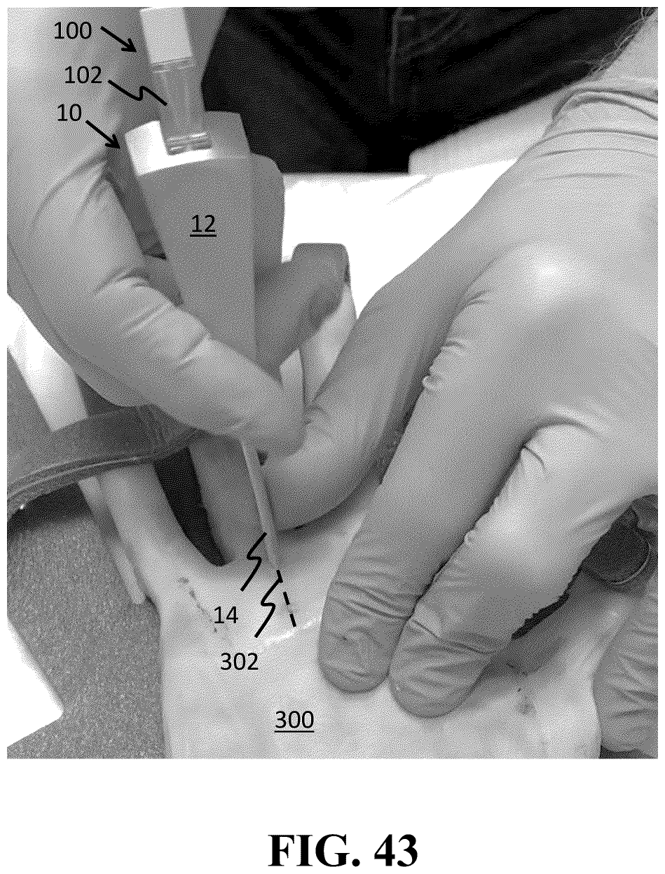

Referring now to FIGS. 42-46, an exemplary method of treatment of a trigger digit using the apparatus 10 is shown, according to some embodiments of the present disclosure. As shown with reference to FIG. 42, a small incision near a tendon sheath 302 may be made in a palm of a hand 300. As shown with reference to FIG. 43, the foot 16 of the apparatus 10 may be inserted within the incision site, such that the foot 16 is seated over the tendon sheath 302. As shown, once the foot 16 has been inserted within the incision site, the foot 16 may not be entirely visible to the surgeon. However, as discussed above, the shapes and contours of the apparatus 10 may aid the surgeon in centering the foot 16 on the tendon sheath 302. As examples, and as further discussed above with reference to FIGS. 27 and 34-36, the equivalent arcs formed by the concave contact surface plane 35 and the rear surface plane 71, as well as the equivalence of the rear surface angle 72 and the contact surface angle 39, may aid the surgeon in centering the foot 16 on the tendon sheath 302. Once the foot 16 has been centered on the tendon sheath 302, the foot 16 may be maneuvered about the tendon sheath 302. For example, and as discussed above with reference to FIG. 20, each of the first and second lips 31, 33 of the foot 16 may sweep soft tissue from opposing sides from the surface of the tendon sheath.

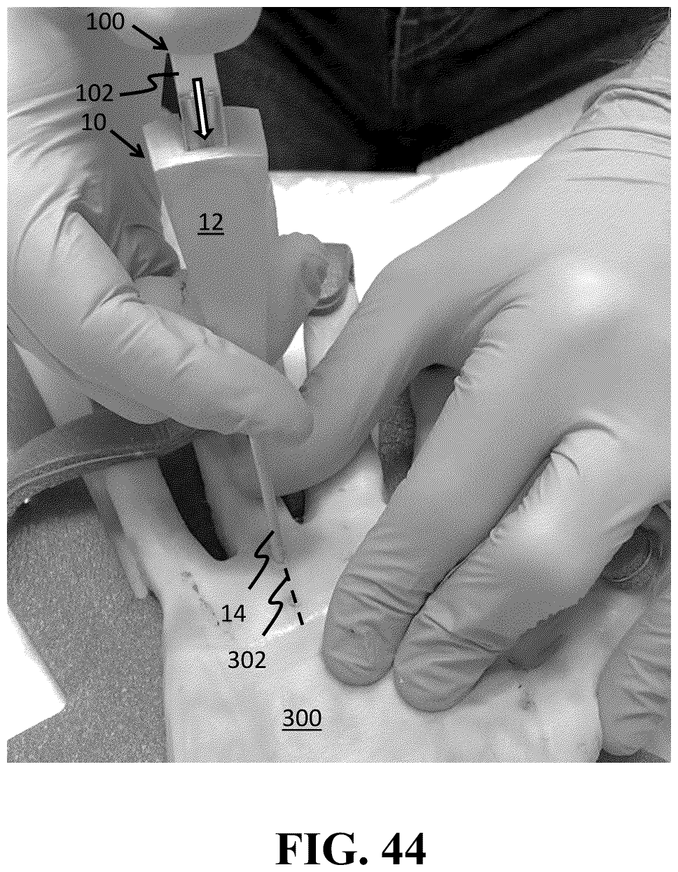

As shown with reference to FIG. 44, the surgeon may then press the hub 102 into the receiver 20 until the hub 102 is seated within the receiver 20 as discussed herein, such that the instrument 100 is further advanced through the channel 24 and the tip 110 projects from the foot 16 to make an incision in the A1 pulley. In some embodiments, and as shown with reference to FIG. 43, when the foot 16 is first inserted within the incision site, the hub 102 may be at least partially received within the receiver 20, such that the tube 104 is positioned within the shaft 14, but the tip 110 has yet to project from the foot 16 (which may occur when the hub 102 is pressed further into the receiver 20 such that the hub 102 is fully seated within the receiver 20). In other embodiments, the instrument 100 is inserted within the apparatus 10 following the aforementioned insertion of the foot 16 within the incision site.

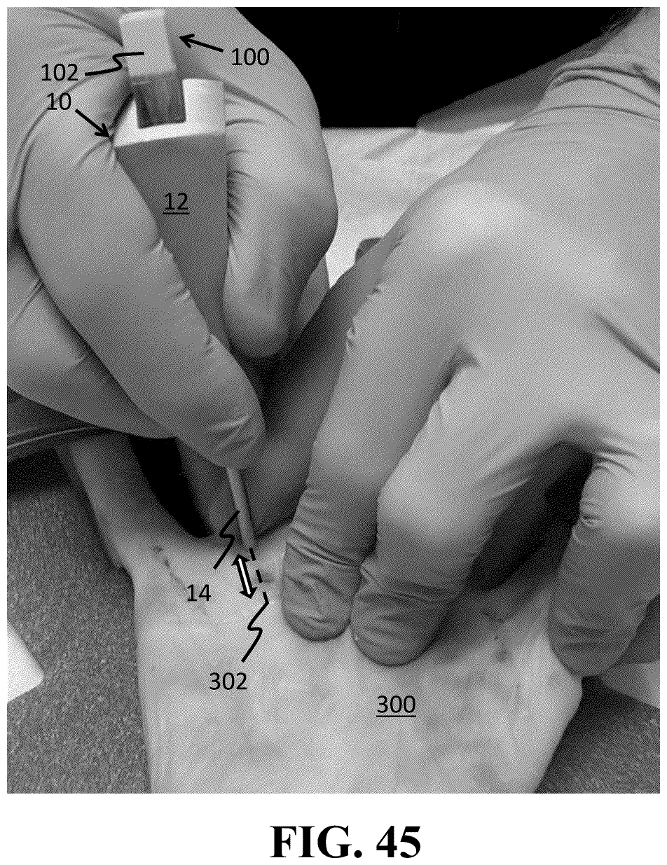

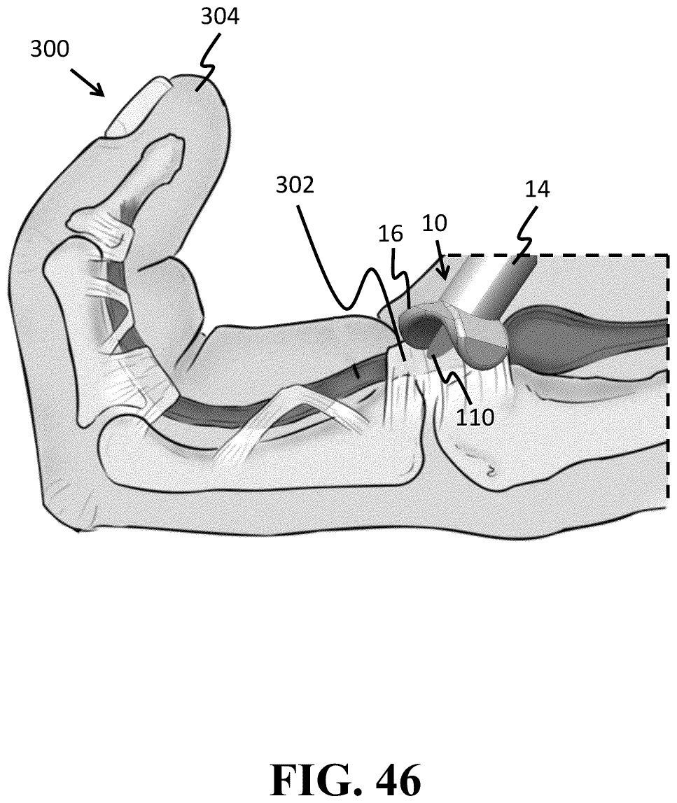

As shown with reference to FIGS. 45 and 46, the tip 110 of the instrument 100 may then be advanced to dissect tissue (e.g., make appropriate cuts on the A1 pulley) by manually controlling the handle in a back and forth or side to side motion. For example, and as shown with particular reference to FIG. 46, the tip 110 of the instrument 100 may be advanced to make appropriate cuts on an A1 pulley 302 of a finger 304 on the hand 300.

Accordingly, the present disclosure further provides for a method. The method may include a first step of providing the handle 12, the shaft 14 extending distally from the handle 12, and the foot 16 disposed on a distal end of the shaft 14 opposite the handle 12. The method may further include a second step of positioning the foot 16 on the tendon sheath 302. As discussed above, the foot 16 may include the concave contact surface 36 and the opening 18 disposed on the concave contact surface 36, where the concave contact surface 36 is shaped to accommodate the tendon sheath 302. The method may further include a third step of advancing the cutting instrument 100 through the channel 24 defined between the port 22 and the opening 18, such that the tip 110 of the cutting instrument 100 projects distally out of the opening 18 and is operable to dissect tissue during a surgical procedure.

Thus, although there have been described particular embodiments of the present invention of a new and useful APPARATUS AND METHODS FOR TRIGGER DIGIT RELEASE, it is not intended that such references be construed as limitations upon the scope of this invention.

What is claimed is:

1. An apparatus, comprising:
a handle;
a shaft extending distally from the handle;
a receiver defined in a proximal end of the handle opposite the shaft, the receiver including a port;
a foot disposed on a distal end of the shaft opposite the handle, the foot including a concave contact surface, and an opening disposed on the concave contact surface, the concave contact surface shaped to accommodate a tendon sheath; and
a channel defined from the port to the opening,
wherein the channel is configured to receive a cutting instrument including a tip that is configured to dissect tissue during a surgical procedure.

2. The apparatus of claim 1, wherein the concave contact surface and a rear surface of the handle on the proximal end of the handle are parallel.

3. The apparatus of claim 2, wherein each of the concave contact surface and the rear surface form a seventy-degree angle relative to a longitudinal axis of the shaft.

4. The apparatus of claim 3, wherein the concave contact surface is curved, such that the concave contact surface forms a first radius of curvature,
wherein the rear surface of the handle forms is curved, such that the rear surface forms a second radius of curvature, and
wherein the first radius of curvature and the second radius of curvature are equivalent.

5. The apparatus of claim 1, wherein the cutting instrument further includes a hub and a tube that is received by the channel, and
wherein the hub becomes seated within the receiver when the channel receives the tube.

6. The apparatus of claim 5, wherein the receiver includes a dimensional profile matching a dimensional profile of the hub, such that the tube is prevented from being rotated within the channel when the hub becomes seated within the receiver.

7. The apparatus of claim 5, wherein opposing lateral ends of the foot extend distally relative to the opening, and
wherein when the hub is seated within the receiver, the tip of the cutting instrument projects distally out of the opening of the foot and is prevented from extending further from the opening than the opposing lateral ends of the foot.

8. A method, comprising:
providing a handle, a shaft extending distally from the handle, and a foot disposed on a distal end of the shaft opposite the handle;
positioning the foot on a tendon sheath, the foot including a concave contact surface and an opening disposed on the contact surface, the concave contact surface shaped to accommodate the tendon sheath;
inserting at least a portion of a cutting instrument into a port on a receiver defined in a proximal end of the handle opposite the shaft; and
advancing the cutting instrument though a channel defined between the port and the opening, such that a tip of the cutting instrument projects distally out of the opening and is operable to dissect tissue during a surgical procedure.

9. The method of claim 8, wherein the concave contact surface and a rear surface of the handle on the proximal end of the handle are parallel.

10. The method of claim 9, wherein each of the concave contact surface and the rear surface form a seventy-degree angle relative to a longitudinal axis of the shaft.

11. The method of claim 10, wherein the concave contact surface is curved, such that the concave contact surface forms a first radius of curvature,
wherein the rear surface of the handle forms is curved, such that the rear surface forms a second radius of curvature, and
wherein the first radius of curvature and the second radius of curvature are equivalent.

12. The method of claim 8, wherein the cutting instrument is advanced through the shaft until a hub of the cutting instrument becomes seated within the receiver.

13. The method of claim 12, wherein the receiver includes a dimensional profile matching a dimensional profile of the hub, such that the cutting instrument is prevented from being rotated within the channel when the hub becomes seated within the receiver.

14. The method of claim 12, wherein opposing lateral ends of the foot extend distally relative to the opening, and
wherein when the hub is seated within the receiver, the tip of the cutting instrument projects distally out of the opening of the foot and is prevented from extending further from the opening than the opposing lateral ends of the foot.

15. An apparatus, comprising:
a handle;
a foot connected to a distal end of the handle, the foot including a concave contact surface, and an opening disposed on the concave contact surface, the concave contact surface shaped to accommodate a tendon sheath;
a receiver defined in a proximal end of the handle opposite the foot, the receiver including a port; and
a channel defined from the port to the opening,
wherein the channel is configured to receive a cutting instrument including a tip that is configured to dissect tissue during a surgical procedure.

16. The apparatus of claim 15, wherein the concave contact surface and a rear surface of the handle on the proximal end of the handle are parallel.

17. The apparatus of claim 16, wherein the concave contact surface is curved, such that the concave contact surface forms a first radius of curvature,
wherein the rear surface of the handle forms is curved, such that the rear surface forms a second radius of curvature, and
wherein the first radius of curvature and the second radius of curvature are equivalent.

18. The apparatus of claim 15, wherein the cutting instrument further includes a hub and a tube that is received by the channel, and
wherein the hub becomes seated within the receiver when the channel receives the tube.

19. The apparatus of claim 18, wherein the receiver includes a dimensional profile matching a dimensional profile of the hub, such that the tube is prevented from being rotated within the channel when the hub becomes seated within the receiver.

20. The apparatus of claim 18, wherein opposing lateral ends of the foot extend distally relative to the opening, and
wherein when the hub is seated within the receiver, the tip of the cutting instrument projects distally out of the opening of the foot and is prevented from extending further from the opening than the opposing lateral ends of the foot.
